(19) United States
(12) Patent Application Publication
(10) Pub. No.: US 2020/0061211 A1
(43) Pub. Date: Feb. 27, 2020

(54) METHODS FOR DELIVERING GENE EDITING REAGENTS TO CELLS WITHIN ORGANS

(71) Applicant: BLUEALLELE, LLC, MAPLE GROVE, MN (US)

(72) Inventor: Nicholas J. Baltes, Maple Grove, MN (US)

(21) Appl. No.: 16/547,019

(22) Filed: Aug. 21, 2019

Related U.S. Application Data
(60) Provisional application No. 62/721,475, filed on Aug. 22, 2018.
(60) Provisional application No. 62/800,664, filed on Feb. 4, 2019.
(60) Provisional application No. 62/859,165, filed on Jun. 9, 2019.

(51) Int. Cl. A61K 48/00; A61M 5/168; A61M 25/01; A61M 25/00

(52) U.S. Cl. CPC A61K 48/0075; A61K 48/0083; C12N 9/22; A61M 25/0158; A61M 25/0026; A61M 5/168

(57) ABSTRACT

The present disclosure is in the field of medical devices and gene editing, particularly the use of medical devices for the targeted delivery of gene editing reagents in vivo or ex vivo. The methods and materials described herein provide control over the location and timing of delivery, along with the ability to deliver gene editing reagents as nucleic acids, virus particles, or protein. Furthermore, the methods and materials can be used to reduce or eliminate the systemic spread of gene editing reagents in non-target tissues/organs. The methods and devices described herein can be used for gene editing in animals.

### REFERENCE TO RELATED APPLICATIONS

This application claims the benefit of previously filed and co-pending applications U.S. Ser. No. 62/721,475 filed Aug. 22, 2018, U.S. Ser. No. 62/800,664 filed Feb. 4, 2019, and U.S. Ser. No. 62/859,165 filed Jun. 9, 2019, the contents of which are incorporated herein by reference in their entirety.

### TECHNICAL FIELD

This document relates to methods for the in vivo and ex vivo genome modification of cells within organs. More specifically, this document relates to the use of medical devices and perfusion methods for targeted delivery of gene editing reagents.

### BACKGROUND

The in vivo modification of genomic DNA in organs using gene editing reagents is an attractive approach for therapy as it avoids ex vivo based methods which require extensive resources and labor. However, in vivo approaches rely on tissue-specific targeting or local delivery and/or target cell-specific gene expression. Progress in delivery has been made in the liver, eye, brain and muscle, primarily using viral vectors such as adeno-associated viruses (AAV) combined with local injection into the parenchyma or systemic intravenous injection, albeit with mixed rates of success.

The liver has been one of the main targets for in vivo gene therapy trials. In initial clinical trial studies for treatment of hemophilia, AAV vectors were used to deliver a gene encoding factor IX; however, expression was limited to only a few weeks (Manno et al., Nature Medicine 12:342-347, 2006). When immunosuppression mechanisms were incorporated into the approach, transgene expression persisted for years and levels of factor IX were increased to 2 to 7% of normal levels (Nathwani et al., New England Journal of Medicine 365:2357-2365, 2011; Nathwani et al., New England Journal of Medicine 371:1994-2004, 2014). Efforts to improve the efficacy of the therapy have used variants of factor IX with high activity, thereby permitting a 4 to 120 times decrease in the levels of AAV particles required (George et al., New England Journal of Medicine 377:2215-2227, 2017). The decrease in AAV particles has been attributed to a lower rate and severity of antivector immune responses.

Additional studies targeting hepatocytes have used AAV to deliver alpha-L-iduronidase gene for mucopolysaccharidosis I (MPS I) and the iduronidate-2-sulfatase gene for mucopolysaccharidosis II (MPS II) along with zinc finger nucleases (ZFNs) designed to integrate the genes into the highly-expressed albumin locus. Delivery of the AAV particles to the liver was achieved by non-localized, systemic intravenous infusion.

The retina of the eye has also been a target in clinical trials and product development. AAV vectors encoding a functional RPE65 gene were delivered to patients via subretinal injection for the treatment of inherited blindness (with the patients having mutations in the RPE65 gene). Following promising results of a phase 3 gene therapy trial (Russell et al., Lancet 390:849-860, 2017), direct injection of AAV is now being pursued in other forms of blindness, including achromatopsia, choroideremia, Leber's hereditary optic neuropathy, X-linked retinoschisis, and X-linked retinitis pigmentosa.

Other in vivo therapies have aimed to treat certain neuromuscular disorders, including adrenoleukodystrophy (ALD), spinal muscular atrophy (SMA), metachromatic leukodystrophy, and aromatic L-amino acid decarboxylase (AADC) deficiency. Both integrating and nonintegrating viral vectors have been used in gene therapy trials.

The safe and effective delivery of therapeutic reagents is an ongoing challenge for gene therapy and gene editing. Development of additional methods for in vivo delivery, particularly those customized for using together with gene editing reagents, with or without the use of viruses, can provide additional approaches for treating genetic disorders, cancer or predispositions to cancer.

### SUMMARY

The systems and methods presented in this document help address several concerns and bottlenecks in the delivery of gene editing reagents to cells in organs. These concerns and bottlenecks include i) safety, ii) efficacy, and iii) access to organs not primarily targeted by viral or non-viral vectors. Regarding safety, a primary concern includes the unintentional or unknowing delivery of gene editing reagents to non-target cells. Once within a non-target cell, the gene editing reagents can potentially create on-target modifications or off-target modification—both of which are undesired or unnecessary and are significant safety concerns. Further, unlike gene therapy, the controlled delivery of gene editing tools is important because permanent cellular changes occur with gene editing (but usually not gene therapy), and these changes can occur with low expression of the gene editing reagents (unlike gene therapy where sustained moderate to strong expression is usually desired). Regarding efficacy, a primary concern for therapeutics using gene editing reagents is that a minimum therapeutic threshold is reached, such that the patient realizes a benefit to the therapy. For many methods which use viral based deliveries, patients can usually only receive one dose of the therapy, making efficacy of the gene editing reagents a primary concern. Further, for non-viral methods, the efficacy is frequently lower than viral based therapies, which may cause challenges overcoming the minimum therapeutic threshold. Finally, regarding access to organs, there are many organs where common delivery tools (e.g., AAV and serotypes) fail to target, or they are at present at low levels and they may infect other cells better in other organs (creating off-target delivery concerns).

The systems and methods presented within this document help address the shortcomings and challenges of delivering gene editing reagents to organs by synergistically combining medical devices with gene editing reagents. The systems and methods presented here include i) a dual-catheter system to precisely delivery gene editing reagents to a target organ and reduce systemic spread of the gene editing reagent exiting the organ, ii) an ex vivo based system which enables controlled delivery of gene editing reagents to an organ connected to a perfusion system, and iii) a single catheter system which deposits gene editing reagents and facilitates cellular uptake of the reagents through the use of accessories (e.g., magnets, electrodes, sonication).

The advantages of the dual-catheter system include the i) controlled dosage and delivery of gene editing reagents to a large number of organs, ii) ability to deliver gene editing reagents in the form of viral or non-viral vectors, and where delivery of viral vectors is not necessary, iii) ability to deliver multiple rounds of gene editing reagents to facilitate reaching the minimum therapeutic threshold, iv) ability to reduce or prevent gene editing reagents from spreading systemically and accessing non-target organs, v) the ability to add accessories to the distal ends of the catheters to facilitate cellular uptake or capture of the gene editing reagents, and vi) provides a protected path to the organ for non-viral gene editing reagents to be protected from nucleases/proteases in the blood. The methods described herein using the dual-catheter system can include choosing a solution comprising at least one gene editing reagent, inserting a first medical device within a lumen that is in proximity to or within said organ, inserting a second medical device within a lumen that is in proximity to or within said organ, and administering said solution through said first medical device. The medical devices can be catheters. The catheters can include an accessory to facilitate delivery or capture of the gene editing reagents, including a balloon, electrode, magnet, needle, or acoustic device. The first catheter for depositing the gene editing reagent can be inserted into an arterial lumen in proximity to or within a target organ. The second catheter for capturing or inactivating the gene editing solution can be inserted into a venous lumen. The target organ can include the liver, pancreas, spleen, gastrointestinal tract, brain, lungs, prostate, eye, prostate, kidney and heart. In a specific embodiment, the organ can be the liver and the first catheter can be inserted into the hepatic artery and the second catheter can be inserted into a hepatic vein or the inferior vena cava. In another embodiment, the organ can be the kidney and the first catheter can be inserted into the renal artery and the second catheter can be inserted into the renal vein. The organ can be from a host including a human, mouse, rat, guinea pig, hamster, dog, pig, sheep, chimpanzee, monkey, horse or cow. The gene editing reagent can be a rare-cutting endonuclease, a transposase, or a donor molecule. More specifically, the gene editing reagent can be CRISPR (e.g., SpCas9), transcription activator-like effector nucleases, zinc-finger nucleases, CRISPR-associated transposases, transposons, or donor molecules. The gene editing reagent can be in the form of protein (e.g., zinc finger nuclease protein), nucleic acid (e.g., Cas9 and gRNA in DNA or RNA format), or virus particles (e.g., Cas9 encoded on an AAV vector and packaged within an AAV particle). The gene editing reagent can be mixed together with carriers, including magnetic nanoparticles or lipid nanoparticles. The methods presented herein can further include delivering an electric pulse, sound energy or magnetic field to the target organ. The methods can further include using a guidewire to facilitate insertion of the catheter within the target lumen. The methods can include using the second catheter to remove or inactivate gene editing reagents leaving the target organ. The second medical device can comprise a balloon and channel, where fluid exiting the organ is collected through the second medical device. In one embodiment, the fluid can be filtered (i.e., gene editing reagents are removed) and then reintroduced into the host. The second catheter can comprise a magnet to help capture gene editing reagents carried on magnetic nanoparticles. In some instances, the second catheter can be used to administer a solution which contains a compound that inactivates the gene editing reagent. The compound can include a DNase, RNase, RNA oligonucleotide, and anti-CRISPR protein. Both the catheters can be guided to their target lumen using a guidewire.

The advantages of the ex vivo based systems described herein include i) controlled dosage and delivery of gene editing reagents, ii) ability to deliver gene editing reagents in the form of viral or non-viral vectors, and where delivery of viral vectors is not necessary, iii) ability to deliver multiple rounds of gene editing reagents to facilitate reaching the minimum therapeutic threshold, and iv) avoids the problem of systemic spread of gene editing reagents, and v) permits introduction of external stimuli (e.g., electricity or magnetic fields) to help facilitate cellular uptake of the gene editing reagents and vi) enables the delivery of gene editing reagents through the vasculature of the organ, or directly into the parenchyma, or both. The methods described herein which use the ex vivo based perfusion systems can include selecting a solution comprising at least one gene editing reagent, isolating or removing an organ from a host, connecting said organ to a perfusion system, perfusing a medical fluid through the organ, and administering said gene editing solution to said organ. The perfusion system can include a peristaltic or centrifugal pump for advancing the medical fluid through the tubing. The perfusion system can further include an oxygenator. The organ can include a liver, pancreas, spleen, gastrointestinal tract, brain, lungs, prostate, eye, kidney and heart. The host can include a human, mouse, rat, guinea pig, hamster, dog, pig, sheep, chimpanzee, monkey, horse or cow. The perfusion system, including the medical fluid and organ, can be stored in hypothermic temperatures (e.g., 4 degrees Celsius), normothermic temperatures (e.g., 37 degrees Celsius), or at room temperature (e.g., approximately 21 degrees Celsius). The medical fluid pumped through the target organ can be Belzer's Gluconate-Albumin solution, University of Wisconsin solution, histidine-tryptophan-ketoglutarate solution, blood, Lifor, or AQIX-RS-I. The medical solution can further comprise an oxygen carrier, including a hemoglobin-based oxygen carrier. The gene editing reagent can be delivered to the target organ through a tube connected to the arterial lumen. The gene editing reagent can be a rare-cutting endonuclease, a transposase, or a donor molecule. More specifically, the gene editing reagent can be CRISPR (e.g., SpCas9), transcription activator-like effector nucleases, zinc-finger nucleases, CRISPR-associated transposases, transposons, or donor molecules. The gene editing reagent can be in the form of protein (e.g., zinc finger nuclease protein), nucleic acid (e.g., Cas9 and gRNA in DNA or RNA format), or virus particles (e.g., Cas9 encoded on an AAV vector and packaged within an AAV particle). The gene editing reagent can be mixed together with carriers, including magnetic nanoparticles or lipid nanoparticles. The methods presented herein can further include delivering an external electric pulse, sound energy or magnetic field to the target organ within the perfusion system. In one example, the gene editing reagents are delivered on magnetic nanoparticles and a magnet is placed next to the organ in the perfusion system.

The advantages of the single-catheter system include the i) controlled dosage and delivery of gene editing reagents to a wide range of organs, ii) ability to deliver gene editing reagents in the form of viral or non-viral vectors, and where delivery of viral vectors is not necessary, iii) ability to deliver multiple rounds of gene editing reagents to facilitate reaching the minimum therapeutic threshold, and iv) the ability to add accessories to the distal ends of the catheters to facilitate cellular uptake or capture of the gene editing reagents and vi) provides a protected path to the organ for non-viral gene editing reagents to be protected from nucleases/proteases in the blood. The methods described herein using the single-catheter system can include choosing a solution comprising at least one gene editing reagent, inserting a medical device within a lumen that is in proximity to or within said organ, and administering said solution through the medical device. The medical device may comprise a catheter, wherein the catheter may further comprise an accessory including an electrode, magnet, needle, or acoustic device. The target organ can include liver, pancreas, spleen, gastrointestinal tract, brain, lungs, prostate, eye, prostate, kidney or heart. The catheter for delivery of at least one gene editing reagent can be inserted into an arterial lumen, which provides fluid to the target organ. The target organ can be the liver and the lumen that the catheter is inserted can be the hepatic artery. The target organ can be the kidney and the lumen that the catheter is inserted can be the renal artery. The target organ can be selected from a host, where the host includes a human, mouse, rat, guinea pig, hamster, dog, pig, sheep, chimpanzee, monkey, horse or cow. The gene editing reagents can include a composition that alters the sequence of DNA. The gene editing reagent can be a rare-cutting endonuclease, a transposase, or a donor molecule. More specifically, the gene editing reagent can be CRISPR (e.g., SpCas9), transcription activator-like effector nucleases, zinc-finger nucleases, CRISPR-associated transposases, transposons, or donor molecules. The gene editing reagent can be in the form of protein (e.g., zinc finger nuclease protein), nucleic acid (e.g., Cas9 and gRNA in DNA or RNA format), or virus particles (e.g., Cas9 encoded on an AAV vector and packaged within an AAV particle). The gene editing reagent can be mixed together with carriers, including magnetic nanoparticles or lipid nanoparticles. The methods presented herein can further include delivering an electric pulse, sound energy or magnetic field to the target organ. The methods can further include using a guidewire to facilitate insertion of the catheter within the target lumen.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF FIGURES

FIG. 1 is illustrations of gene editing catheters (GECs) and accessories for delivering gene editing reagents to organs-of-interest. 39, distal end of delivery catheter; 40, distal end of catheter, reagent dispenser; 41, distal end of delivery catheter with accessory; 42, general position for electrode, sonicator or magnet; 43, distal end of delivery catheter with needle; 63, needle; 64, distal end of catheter with a multi-needle array.

FIG. 2 is an illustration showing the general locations for placement of the GEC. 44, organ-of-interest (target organ); 45, arterial lumen; 46, directional flow of fluid; 47, gene-editing catheter; 48, proximity; 49, intra-organ or within the organ.

FIG. 3 is illustrations of safety gene editing catheters (S-GECs) and accessories for capturing or inactivating gene editing reagents leaving the organ-of-interest. 50, balloon that presses against lumen wall; 51, directional flow of fluid; 52, Exit for fluid; 53, distal end of S-GEC with binding elements; 54, distal end of S-GEC with collection tube; 55, distal end of S-GEC with magnet; 56, distal end of S-GEC with magnet; 57, distal end of S-GEC with reagent dispenser.

FIG. 4 is an illustration showing the general locations for placement of the S-GEC. 44, organ-of-interest (target organ); 45, arterial lumen; 46, directional flow of fluid; 48, proximity; 49, intra-organ or within the organ; 48, proximity; 49, intra-organ or within the organ; 60, safety gene editing catheter.

FIG. 5 is an illustration showing the general locations for placement of S-GECs. 44, organ-of-interest (target organ); 45, arterial lumen; 46, directional flow of fluid; 48, proximity; 49, intra-organ or within the organ; 58, downstream after branching; 60, safety gene editing catheter.

FIG. 6 is an illustration of the general process for using GEC and S-GEC devices to deliver gene editing reagents to an organ. 44, organ-of-interest (target organ); 46, directional flow of fluid; 47, gene-editing catheter; 59, gene editing reagent dispensed; 60, safety gene editing catheter; 61, gene editing reagent.

FIG. 7 is an illustration showing the general use of a single-component gene editing catheters (SS-GEC). 44, organ-of-interest (target organ); 46, directional flow of fluid; 59, gene editing reagent dispensed; 61, gene editing reagent; 62, single-component gene editing catheter.

FIG. 8 is an illustration of a liver showing the positioning of the S-GEC and GEC. The S-GEC is positioned in the inferior vena cava following the connections of the hepatic veins. The GEC is positioned in the hepatic artery. 47, gene-editing catheter; 60, safety gene editing catheter; 65, hepatic veins; 66, inferior vena cava; 67, portal vein.

FIG. 9 is an illustration of the blood vessels supplying a pancreas, along with the position of a GEC. 47, gene-editing catheter; 69, right gastro-omental artery; 70, superior pancreaticoduodenal artery (SPDA); 71, splenic artery; 72, posterior SPDA; 73, anterior SPDA; 74, anterior IPDA; 75, inferior pancreaticoduodenal artery (IPDA); 76, posterior IPDA; 77, dorsal pancreatic artery; 78, greater pancreatic artery.

FIG. 10 is an illustration of the ducts within the pancreas, along with the position of a GEC. 64, distal end of catheter with a multi-needle array; 79, Main pancreatic duct; 80, Bile duct; 81, Accessory pancreatic duct; 82, Major duodenal papilla.

FIG. 11 is an illustration of the arteries and veins entering or exiting the spleen, along with the position of a GEC and S-GEC. 47, gene-editing catheter; 83, Splenic artery; 84, Splenic vein.

FIG. 12 is an illustration of the blood vessels supplying a gastrointestinal tract, along with the position of a GEC. 47, gene-editing catheter; 85, superior mesenteric artery; 86, inferior pancreaticoduodenal artery; 87, inferior mesenteric artery.

FIG. 13 is an illustration of a GEC and S-GEC with magnets which create a magnetic field around an organ. 41, distal end of delivery catheter with accessory; 56, distal end of S-GEC with magnet.

FIG. 14 is an illustration of S-GECs with magnets for capturing magnetic nanoparticles exiting an organ. 88, magnets with chambers; 89, flow of fluid; 90, lumen wall; 91, catheter wall; 92, magnet; 93, diametrically magnetized ring.

FIG. 15 is an illustration of S-GECs with collection tubes for removing fluids exiting an organ. 89, flow of fluid; 90, lumen wall; 94, discard or sent through dialysis machine; 95, blood transfusion delivery; 96, fluid collection, discarded or sent through dialysis machine.

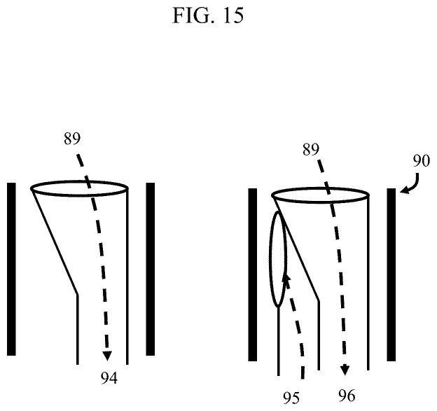

FIG. 16 is a schematic of the perfusion circuit. Individual components are listed below the schematic. 1, reservoir; 2, arterial line; 3, peristaltic pump; 4, y-connector; 5, inlet for delivery catheter or gene editing deposition; 6, arterial line; 7, organ chamber; 8, barb connector and securing clamps; 9, venous line; 10, y-connector; 11, inlet for capturing catheter; 12, three-way stop valve; 13, reservoir; 14, venous line.

FIG. 17 is an illustration of the catheter combinations 1, 2 and 3 for delivery and capture of gene editing reagents.

FIG. 18 is an illustration of a general gene editing catheter for navigating to the target lumen and delivering gene editing reagents. 15, general delivery catheter or capturing catheter; 16, catheter hub; 17, proximal region; 18, guide wire; 19, distal region; 20, distal end; 21, main shaft, catheter body; 22, proximal end.

FIG. 19 are two graphs showing the relative mean average intensity of images of kidney and liver tissue stained in trypan blue. Y-axis is the normalized relative mean average intensity of the tissue; X-axis is the time in hours post removal of organs from the host.

FIG. 20 are images of PCR gels detecting gene editing reagents. 97, negative control; 98, liver, combination 1 near magnet, sample 1; 99, liver, combination 1 near magnet, sample 2; 100, liver, combination 1 near magnet, sample 3; 101, liver, combination 1 neighboring magnet; 102, liver, combination 2 neighboring electrode; 103, liver, combination 2 near electrode; 104, kidney, external electrode, neighboring electrode; 105, kidney, external electrode, sample 1; 106, kidney, external electrode, sample 2; 107, kidney, external electrode, sample 3; 108, kidney, external electrode, sample 4; 109, kidney, external electrode, sample 5; 110, kidney, external electrode, sample 6; 111, kidney, external electrode, sample 7; 112, kidney, external electrode, sample 8; 113, kidney, external electrode, sample 9; 114, liver, combination 2, magnet; 115, perfusion system, fluid within chamber, sample 1; 116, perfusion system, fluid within chamber, sample 2; 117, perfusion system, fluid captured by collection catheter combination 1, sample 1; 118, perfusion system, fluid captured by collection catheter combination 1, sample 2; 119, perfusion system, fluid within chamber, sample 3; 120, perfusion system, fluid captured by collection catheter combination 1, sample 3; 121, perfusion system, fluid captured by collection catheter combination 1, sample 4; 122, perfusion system, magnet; 123, positive control (purified plasmid DNA).

FIG. 21 are images of gels detecting gene editing (˜10 kb deletion) and internal controls (WT KIT gene). 124, liver, combination 1 near magnet, sample 1; 125, liver, combination 1 near magnet, sample 2; 126, liver, combination 1 near magnet, sample 3; 127, liver, combination 1 neighboring magnet (no induction); 128, kidney, external electrode, neighboring electrode (no induction); 129, kidney, external electrode, sample 1; 130, kidney, external electrode, sample 2; 131, kidney, external electrode, sample 3; 132, kidney, external electrode, sample 4; 133, kidney, external electrode, sample 5; 134, kidney, external electrode, sample 6; 135, kidney, external electrode, sample 7; 136, kidney, external electrode, sample 8; 137, kidney, external electrode, sample 9; 138, PCR negative control (no DNA).

FIG. 22 is an image of a gel detecting the presence of gene editing tools in fluid captured by a collection catheter. 139, 1 kb ladder; 140, 1 ul of sample 1; 141, 10 ul of sample 1; 142, 1 ul of sample 2; 143, plasmid DNA positive control; 144, no DNA control.

### DETAILED DESCRIPTION

This present document is based in part on the discovery of methods and materials for targeted delivery of gene editing reagents to cells within organs. The methods and materials described herein provide control over the location and timing of delivery, along with the ability to deliver gene editing reagents as nucleic acids, virus particles, or protein. Furthermore, the methods and materials can be used to reduce or eliminate the systemic spread of gene editing reagents in non-target tissues/organs. The methods and devices described herein help enable targeted, efficient and safe gene editing in animals.

The present invention is directed to a method to deliver gene editing reagents to cells in an organ. The method can comprise choosing a solution comprising at least one gene editing reagent, inserting a medical device within a lumen that is in proximity to or within said organ, and administering said solution through the medical device. The medical device may comprise a catheter, wherein the catheter may further comprise an accessory including an electrode, magnet, needle, or acoustic device. The target organ can include liver, pancreas, spleen, gastrointestinal tract, brain, lungs, prostate, eye, prostate, kidney or heart. The catheter for delivery of at least one gene editing reagent can be inserted into an arterial lumen, which provides fluid to the target organ. The target organ can be the liver and the lumen that the catheter is inserted can be the hepatic artery. The target organ can be the kidney and the lumen that the catheter is inserted can be the renal artery. The target organ can be selected from a host, where the host includes a human, mouse, rat, guinea pig, hamster, dog, pig, sheep, chimpanzee, monkey, horse or cow. The gene editing reagents can include a composition that alters the sequence of DNA. The gene editing reagent can be a rare-cutting endonuclease, a transposase, or a donor molecule. More specifically, the gene editing reagent can be CRISPR (e.g., SpCas9), transcription activator-like effector nucleases, zinc-finger nucleases, CRISPR-associated transposases, transposons, or donor molecules. The gene editing reagent can be in the form of protein (e.g., zinc finger nuclease protein), nucleic acid (e.g., Cas9 and gRNA in DNA or RNA format), or virus particles (e.g., Cas9 encoded on an AAV vector and packaged within an AAV particle). The gene editing reagent can be mixed together with carriers, including magnetic nanoparticles or lipid nanoparticles. The methods presented herein can further include delivering an electric pulse, sound energy or magnetic field to the target organ. The methods can further include using a guidewire to facilitate insertion of the catheter within the target lumen.

The present invention is directed to a method for delivering and capturing gene editing reagents in a target organ. The method can include choosing a solution comprising at least one gene editing reagent, inserting a first medical device within a lumen that is in proximity to or within said organ, inserting a second medical device within a lumen that is in proximity to or within said organ, and administering said solution through said first medical device. The medical devices can be catheters. The catheters can include an accessory to facilitate delivery or capture of the gene editing reagents, including a balloon, electrode, magnet, needle, or acoustic device. The first catheter for depositing the gene editing reagent can be inserted into an arterial lumen in proximity to or within a target organ. The second catheter for capturing or inactivating the gene editing solution can be inserted into a venous lumen. The target organ can include the liver, pancreas, spleen, gastrointestinal tract, brain, lungs, prostate, eye, prostate, kidney and heart. In a specific embodiment, the organ can be the liver and the first catheter can be inserted into the hepatic artery and the second catheter can be inserted into a hepatic vein or the inferior vena cava. In another embodiment, the organ can be the kidney and the first catheter can be inserted into the renal artery and the second catheter can be inserted into the renal vein. The organ can be from a host including a human, mouse, rat, guinea pig, hamster, dog, pig, sheep, chimpanzee, monkey, horse or cow. The gene editing reagent can be a rare-cutting endonuclease, a transposase, or a donor molecule. More specifically, the gene editing reagent can be CRISPR (e.g., SpCas9), transcription activator-like effector nucleases, zinc-finger nucleases, CRISPR-associated transposases, transposons, or donor molecules. The gene editing reagent can be in the form of protein (e.g., zinc finger nuclease protein), nucleic acid (e.g., Cas9 and gRNA in DNA or RNA format), or virus particles (e.g., Cas9 encoded on an AAV vector and packaged within an AAV particle). The gene editing reagent can be mixed together with carriers, including magnetic nanoparticles or lipid nanoparticles. The methods presented herein can further include delivering an electric pulse, sound energy or magnetic field to the target organ. The methods can further include using a guidewire to facilitate insertion of the catheter within the target lumen. The methods can include using the second catheter to remove or inactivate gene editing reagents leaving the target organ. The second medical device can comprise a balloon and lumen, where fluid exiting the organ is collected through the second medical device. In one embodiment, the fluid can be filtered (i.e., gene editing reagents are removed) and then reintroduced into the host. The second catheter can comprise a magnet to help capture gene editing reagents carried on magnetic nanoparticles. In some instances, the second catheter can be used to administer a solution which contains a compound that inactivates the gene editing reagent. The compound can include a DNase, RNase, RNA oligonucleotide, and anti-CRISPR protein. Both the catheters can be guided to their target lumen using a guidewire.

The present invention is also directed to an ex vivo method for delivering gene editing reagents to cells in an organ. The method can include selecting a solution comprising at least one gene editing reagent, isolating or removing said organ from a host, connecting said organ to a perfusion system, perfusing a medical fluid through the organ, and administering said gene editing solution to said organ. The perfusion system can include a peristaltic or centrifugal pump for advancing the medical fluid through the tubing. The perfusion system can further include an oxygenator. The organ can include a liver, pancreas, spleen, gastrointestinal tract, brain, lungs, prostate, eye, kidney and heart. The host can include a human, mouse, rat, guinea pig, hamster, dog, pig, sheep, chimpanzee, monkey, horse or cow. The perfusion system, including the medical fluid and organ, can be stored in hypothermic temperatures (e.g., 4 degrees Celsius), normothermic temperatures (e.g., 37 degrees Celsius), or at room temperature (e.g., approximately 21 degrees Celsius). The medical fluid pumped through the target organ can be Belzer's Gluconate-Albumin solution, University of Wisconsin solution, histidine-tryptophan-ketoglutarate solution, blood, Lifor, or AQIX-RS-I. The medical solution can further comprise an oxygen carrier, including a hemoglobin-based oxygen carrier. The gene editing reagent can be delivered to the target organ through a tube connected to the arterial lumen. The gene editing reagent can be a rare-cutting endonuclease, a transposase, or a donor molecule. More specifically, the gene editing reagent can be CRISPR (e.g., SpCas9), transcription activator-like effector nucleases, zinc-finger nucleases, CRISPR-associated transposases, transposons, or donor molecules. The gene editing reagent can be in the form of protein (e.g., zinc finger nuclease protein), nucleic acid (e.g., Cas9 and gRNA in DNA or RNA format), or virus particles (e.g., Cas9 encoded on an AAV vector and packaged within an AAV particle). The gene editing reagent can be mixed together with carriers, including magnetic nanoparticles or lipid nanoparticles. The methods presented herein can further include delivering an external electric pulse, sound energy or magnetic field to the target organ within the perfusion system. In one example, the gene editing reagents are delivered on magnetic nanoparticles and a magnet is placed next to the organ in the perfusion system. In another example, the gene editing reagents are delivered to the organ through the perfusion system or through direct injection, and an electric pulse is delivered to the organ.

Also provided herein is a kit, which may be used to deliver genome editing reagents to organs in a human or animal. The kit can comprise a solution containing at least one gene editing reagent, a catheter and instructions for using said catheter and solution. Instructions included in the kit may be affixed to packaging material or may be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions. In another embodiment, the kit can include a solution containing at least one gene editing reagent, a first and second catheter, and instructions for using said first and second catheters and solution. In another embodiment, the kit can include a solution containing at least one gene editing reagent, a catheter, a guidewire and instructions for using said catheter, said guidewire and solution. In another embodiment, the kit can include a solution containing at least one gene editing reagent, a first and second catheter, a guide wire, and instructions for using said first and second catheters and solution. In another embodiment, the kit can include a solution containing at least one gene editing reagent, a first and second catheter, a guidewire, and instructions for using said first and second catheters, said guidewire and solution. In another embodiment, the kit can include a first solution containing at least one gene editing reagent, a first and second catheter, a second solution comprising at least one component to inactivate or destroy said gene editing reagent, and instructions for using said first and second catheters with said first and second solutions. The second solution can include a component such as a restriction endonuclease, DNase, RNase, RNA oligonucleotide, or anti-CRISPR protein. In another embodiment, the kit can include a first solution containing at least one gene editing reagent, a first and second catheter, a second solution comprising at least one component to inactivate or destroy said gene editing reagent, a guidewire and instructions for using said first and second catheters with said first and second solutions and guidewire. The catheter kit can contain instructions to direct a user to (i) insert the catheter into a lumen in proximity to or within an organ; (ii) deliver the at least one gene editing reagent to the organ through the catheter; and, optionally, (iii) activating an accessory to effect uptake of the at least one gene editing reagent by cells in the organ.

In one aspect, this document provides methods for the delivery of gene editing reagents to cells in an organ. The method can include preparing a solution containing at least one gene editing reagent, inserting a medical device within a lumen that is in proximity to or within the organ-of-interest, and administering the solution through a medical device. The medical device can be a catheter. The gene editing reagent can be CRISPR, transcription activator-like effector nucleases (TALENs), or zinc-finger nucleases (ZFNs). The organ-of-interest can be the liver, pancreas, spleen, gastrointestinal tract, brain, lungs, prostate, eye, prostate, kidney or heart. The organ-of-interest can be an organ within a mouse, rat, guinea pig, hamster, dog, pig, sheep, chimpanzee, monkey, horse or cow. The organ can be the liver, and the catheter can deposit the gene editing reagent in the hepatic artery or portal vein. The organ can be the prostate and the catheter can deposit the gene editing regent within the prostrate by going through the lumen wall of the urethra. To facilitate localization and/or cellular uptake of gene editing reagents, the catheter can be outfitted with an electrode, magnet, needle or acoustic device.

In another aspect, this document provides a method to localize gene editing reagents within an organ. The method can include using a catheter to dispense a gene editing reagent in combination with a second medical device to collect or inactivate the gene editing reagent exiting the organ. The second medical device can be a catheter. The catheter can have an accessory including a collection tube, magnet, reagent dispenser, or binding elements.

In a further aspect, this document provides a method to reduce the growth of cancerous cells. The method can include preparing a solution containing a gene editing reagent, inserting a medical device within a lumen that is in proximity to or within the cancerous cells, and administration of the solution through the medical device. The gene editing reagent can be the CRISPR Cas13b system. The cancerous cells can be prostate cancer cells.

In another aspect, this document provides a method to deliver gene editing reagents to cells in an organ, where the method includes preparing a solution comprising at least one gene editing reagent, isolating or removing said organ from a host, connecting said organ to a perfusion system and perfusing a medical fluid through the organ, and administering said gene editing solution to said organ. In one embodiment, this document provides methods for the localization of genome editing reagents within a target organ. The methods can include the use of a catheter which is inserted into lumens, including blood vessels, ducts, or the gastrointestinal tract. The catheters described herein can be customized and tailored for the delivery of gene editing reagents within targeted organs.

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolfe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.), Academic Press, San Diego, 1999; and METHODS IN MOLECULAR BIOLOGY, Vol. 119, “Chromatin Protocols” (P. B. Becker, ed.) Humana Press, Totowa, 1999.

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The terms “polypeptide,” “peptide” and “protein” are used interchangeably to refer to a polymer of amino acid residues. The term also applies to amino acid polymers in which one or more amino acids are chemical analogues or modified derivatives of a corresponding naturally-occurring amino acids.

As used herein, an “endogenous” molecule is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid. Additional endogenous molecules can include proteins, for example, transcription factors and enzymes.

A “gene,” for the purposes of the present disclosure, includes a DNA region encoding a gene product (see infra), as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

The term “gene editing reagent” refers to a reagent, molecule or substance that can alter the sequence of DNA in a cell, or a nucleic acid that encodes for a reagent, molecule or substance that can alter the sequence of DNA in a cell. The gene editing reagent can be a rare-cutting endonuclease, including a meganuclease, a zinc finger nuclease, a TAL effector endonuclease, or a CRISPR endonuclease, or nucleic acid molecules (e.g., viral vectors, plasmid DNA, RNA) coding for such. The gene editing reagent can be a transposase, including the Sleeping Beauty transposase or a CRISPR-associated transposase (Strecker et al., Science 365:48-53, 2019). The gene editing reagent can include nucleic acid molecules designed to be integrated into the DNA in a cell. The nucleic acid molecule can be a donor molecule. The donor molecule can be used by the cell as a template for repair of a double-strand break. Information within the donor molecule that differs from the genomic sequence at or near the double-strand break can be stably incorporated into the cell's genomic DNA. Alternatively, a donor molecule can comprise little to no homology to the genomic target site but can harbor elements that facilitate integration into the genome by the non-homologous end joining pathway. These elements can include exposed single stranded or double-stranded DNA ends, or target sites for cleavage by a rare-cutting endonuclease.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

The term “inserting” refers to putting something inside something else. To insert a medical device within a lumen refers to placing a medical device within the outer boundaries of the lumen. In one example, inserting a medical device in a target lumen may be achieved by the Seldinger technique. More specifically, inserting a medical device can be achieved by puncturing a desired vessel or cavity with a sharp hollow needle, advancing a guide wire through the lumen of the needle, advancing the guide wire to the target lumen, and then advancing a catheter over the guidewire to the target lumen in the patient. The term “medical device” refers to an instrument, apparatus, implement, machine, contrivance, implant intended for use in the diagnosis of disease or other conditions, or in the cure, mitigation, treatment, or prevention of disease, in man or other animals, or intended to affect the structure or any function of the body of man or other animals. As described herein, the medical device can be a catheter, a sheath, J wire, syringe, needle, or guidewire. Additionally, the medical device may comprise an electrode, magnet or acoustic accessory.

The term “arterial lumen” refers to a lumen through which blood or fluid travels to a reach a target organ or tissue. The arterial lumen can carry nutrients, oxygenated blood or a fluid to an organ. The arterial lumen can be, but not limited to, the hepatic artery, hepatic portal vein, renal artery, pulmonary artery, splenic artery, ophthalmic artery, central retinal artery, celiac artery, superior mesenteric artery, inferior mesenteric artery, left common carotid artery, or right common carotid artery. The term “venous lumen” refers to a lumen through which blood or fluid travels away from a target organ or tissue. The venous lumen can carry nutrients, deoxygenated blood, or fluid away from an organ. The venous lumen can be, but not limited to, the hepatic veins, inferior vena cava, pulmonary vein, renal vein, splenic vein, central renal vein, internal jugular vein, or external jugular vein.

The term “catheter” refers to a medical device for insertion into canals, vessels, lumens, passageways, or body cavities. A catheter can be a thin, flexible tube made from medical grade materials, including silicone rubber, nylon, polyurethane, polyethylene terephthalate (PET), latex, and thermoplastic elastomers. The catheters described herein may comprise an elongated catheter body, a catheter hub, a distal end region, a proximal end region and optionally a guidewire exit port. For delivery of gene editing reagents, the catheters may comprise a hollow channel.

The term “in proximity to or within” an organ refers to locations within or adjacent to an organ-of-interest. The term “within an organ” refers to a location within a lumen, where the lumen is surrounded by or neighboring parenchymal cells within the organ-of-interest. The term “proximity” is defined herein as a location within a lumen, where the lumen is nearby the organ-of-interest. Further, regarding the arterial side of the organ, proximity refers to a location that is within a lumen, where the fluid within the lumen is flowing towards the organ-of-interest. Proximity further refers to a location within a lumen, where there is no additional branching of the lumen before reaching the organ-of-interest. Regarding the venous side of the organ, proximity refers to a location that is within a lumen, where the fluid within the lumen is flowing away from the organ-of interest.

The term “isolating or removing” as described herein refers to separating something from other things from which they are connected or mixed. When referring to organs, “removing” refers to separating an organ from a host, which includes uncoupling vasculature. “Removing” an organ can refer to complete removal of an organ from a host, including the uncoupling of all sources of vasculature. “Isolating” can refer to the uncoupling of a subset vasculature while the organ remains within the host.

The term “body part” refers to any part of an organism, such as an organ, cavity or extremity. A body part in fluid communication with a target body part, by means of example, can be an entrance to the target body part, an exit to the target body part, a passageway, canal, vessel, artery, lumen, body cavity or other body part in fluid communication with the target body part.

The term “guidewire” refers to a medical device that is used to enter tight spaces within the body. The guidewire can be a flexible wire or spring used as a guide for placement of a larger device or prosthesis, such as a catheter. The guidewire acts as a track for the catheter to pass over to reach a target location within the vessel.

The term “transposase” as used herein refers to one or more proteins that facilitate the integration of a transposon. A transposase can include a CRISPR-associated transposase (Strecker et al., Science 10.1126/science.aax9181, 2019; Klompe et al., Nature, 10.1038/s41586-019-1323-z, 2019). The transposases can be used in combination with a transgene (i.e., a transposon) comprising a transposon left end and right end. The CRISPR transposases can include the TypeV-U5, C2C5 CRISPR protein, Cas12k, along with proteins tnsB, tnsC, and tniQ. In some embodiments, the Cas12k can be from Scytonema hofmanni or Anabaena cylindrica. Alternatively, the CRISPR transposase can include the Cas6 protein, along with helper proteins including Cas7, Cas8 and TniQ.

The terms “left end” and “right end” as used herein refers to a sequence of nucleic acids present on a transposon, which facilitates integration by a transposase. By way of example, integration of DNA using ShCas12k can be facilitated through a left end and right end sequence flanking a cargo sequence (Strecker et al., Science 10.1126/science.aax9181, 2019).

In one embodiment, this document provides methods for delivering and capturing gene editing reagents using catheters. The catheters described herein may comprise an elongated catheter body, a catheter hub, a distal end region, a proximal end region and, optionally, a guidewire port. For delivery of gene editing reagents, the catheters may comprise a hollow catheter channel or a structure on the distal end region for storing liquids or gels.

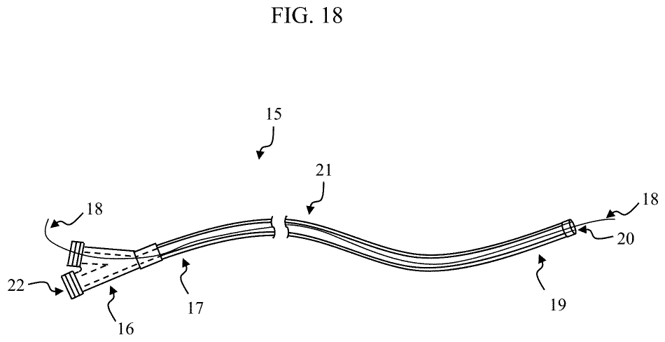

Referring to FIG. 18, a perspective view of a general delivery catheter or capturing catheter is illustrated. The catheter is comprised of elongate tubular member (21; main shaft, catheter body) having distal (20) and proximal ends (22). A hub or other connecting device (16) is present on the proximal end of the device. Port for guidewire (18) or delivery/capture of gene editing reagents is present on the proximal end of the device. For delivery catheters, gene editing reagents can be dispensed through the hub, a channel in the main shaft (21) and exit the distal end (20). For capturing catheters which remove fluid exiting an organ, the fluid can be collected through the distal end of the catheter (20), and traverse through a channel in the main shaft (21) and exit through a port in the hub (16). Accessories such as a balloon, magnet, or electrode or other structure, is present on the distal end of the device. The elongate tubular member (21) can includes at least a guidewire channel, and may contain other channels such as balloon inflation channel, aspiration channel, pull/push wire channels, fluid dispensing channels, fluid collection channels, or any other elongate structures required to deliver or capture the gene editing reagents (3) or other desired functions of the device.

The catheter body may be introduced into a blood vessel or lumen with the guidewire passing through the common channel of the distal region and a first channel of the proximal region. After the catheter body is in place, the movable guidewire may be retracted within the first channel of the distal region and the work element advanced into the common channel from a second channel in the proximal region.

The overall dimensions of the catheter will depend on use, with the length typically being between about 40 cm and 150 cm, usually being between about 40 cm and 120 cm for peripheral catheters and being between about 110 cm and 150 cm for coronary catheters. The catheter body may be composed of a wide variety of biologically compatible materials, including natural or synthetic polymers such as silicone rubber, natural rubber, polyvinyl chloride, polyurethanes, polyesters, polyethylene, polytetrafluoroethylene (PTFE), and the like. The catheter body may be formed as a composite having a reinforcement material incorporated within the elastomeric body in order to enhance strength, flexibility, and toughness. Suitable enforcement layers include wire mesh layers. The flexible tubular members of the catheter body will normally be formed by extrusion, with one or more integral channels being provided. The catheter diameter can then be modified by heat expansion and shrinkage using conventional techniques. Particular techniques for forming the vascular catheters of the present invention are well described in the patent and medical literature.

The catheter body may be formed from a single tubular member, which extends the entire distance from the proximal end to the distal end, or it may be formed from two or more tubular members which are joined together, either in tandem or in parallel. For catheter bodies formed from a single tubular member, the proximal region will be expanded relative to the distal region and appropriate channels will be formed in the interiors of the two regions. Alternatively, the distal region in the catheter body may be formed from a single tubular member having a single channel while the proximal region is formed from a second tubular member having at least two axial channels. The two regions may then be joined together so that the common channel and the distal tubular element is contiguous with both the parallel axial channels and the proximal region. As a second alternative, the catheter body may include a single tubular member having a single axial channel which extends the entire length from the distal end to the proximal end. The proximal section is formed by securing a second tubular member to the side of the first tubular member and penetrating the first tubular member so that the respective channels are made contiguous. The distal region of the catheter is that portion which remains forward of the point where the two tubes are joined.

The distal region of the catheter will typically have a length in the range from about 1 cm to 30 cm, more typically being in the range from about 2 cm to 20 cm, with the proximal region extending in the proximal direction from the distal region. The proximal region, however, need not extend the entire distance to the proximal end of the catheter body. It will often be desirable to extend the guidewire channel formed by the proximal region only a portion of the distance from the distal region back toward the proximal end of the catheter body, typically extending from about 10 cm to 30 cm, more typically extending from 15 cm to 25 cm. In this way, the guidewire channel can have a “monorail” design which facilitates exchange in the catheter over the guidewire. Such monorail designs are described generally in U.S. Pat. No. 4,748,982, the disclosure of which is incorporated herein by reference in its entirety for all purposes.

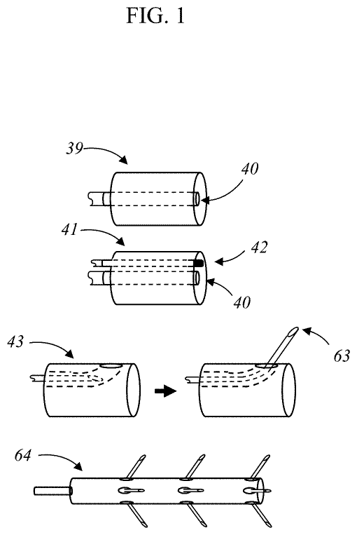

The catheter that positions and dispenses gene editing reagents is herein referred to as a gene editing catheter, or alternatively, a GEC. A GEC can comprise medical grade materials which form a flexible, thin tube. The tube can be inserted through openings or lumens within the subject. The GEC can be outfitted with one or more devices which facilitate dispensing of the gene editing reagents (FIG. 1). The GEC can comprise a reagent dispenser which can store, carry, or dispense liquids or gels. The reagent dispenser can be a flexible tube with an opening at the end of the catheter. The solution comprising gene editing reagents can be stored outside the subject, and when the catheter is properly positioned, the solution can be administered. Alternatively, the reagent dispenser can be an encapsulated device within the catheter, and when the catheter is properly positioned, the dispenser can open and release the solution comprising gene editing reagents.

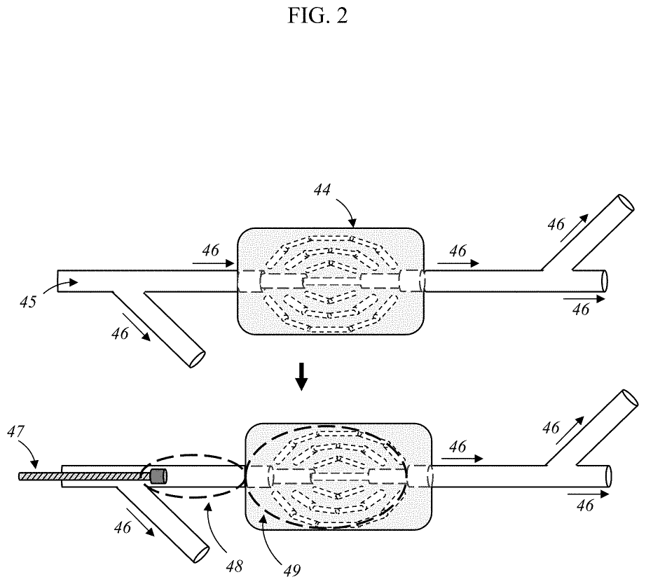

In another embodiment, this document provides methods for positioning the GEC. In general, for the targeted delivery of gene editing reagents to a single organ, the GEC can be positioned at two locations: intra-organ (or alternatively, “within the organ”) or proximity (FIG. 2). Intra-organ, or within the organ, is defined herein as a location within a lumen, where the lumen is surrounded by or neighboring parenchymal cells within the organ-of-interest. Proximity is defined herein as a location within a lumen, where the lumen is nearby the organ-of-interest. With regard to the arterial side, proximity refers to a location that is within a lumen, where the lumen fluid is flowing towards the organ-of-interest. Proximity further refers to a location within a lumen, where there is no additional branching of the lumen before reaching the organ-of-interest. Positioning the GEC at either the proximity or intra-organ position, followed by dispensing of a solution comprising a gene editing reagent, results in the targeted delivery of reagents within the organ-of-interest.

The gene editing reagent dispensed by the GEC can be in the form of a nucleic acid or protein. Gene editing reagents can be in the form of double-stranded or single-stranded DNA (e.g., donor molecules or transgenes), mRNA, RNA (e.g., guide RNA for CRISPR systems,) protein, or an RNA/protein mixture (e.g. CRISPR ribonucleoproteins). The gene editing reagent can be conjugated or associated with a reagent that facilitates stability or cellular update. The reagent can be lipids, calcium phosphate, cationic polymers, DEAE-dextran, dendrimers, polyethylene glycol (PEG) cell penetrating peptides, gas-encapsulated microbubbles or magnetic beads. If delivered as a nucleic acid, the gene editing reagent can be incorporated into a viral particle. The virus can be retroviral, adenoviral, adeno-associated vectors, herpes simplex, pox virus, hybrid adenoviral vector, epstein-bar virus, lentivirus, or herpes simplex virus. The solution comprising the gene editing reagent can be room temperature, or the solution can be cooled to a temperature below 22° C. The solution can be 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., or 22° C. The solution comprising the gene editing reagent can be at 37° C. or a temperature between 37° C. and 22° C. A conditioning fluid, not comprising a gene editing reagent, can be deposited prior to the delivery of gene editing reagents. The conditioning fluid can comprise reagents that prepare the cells in a target organ for transfection. The conditioning fluid can cool the target cells by comprising a liquid at a temperature below 36° C. The fluid can comprise PEG.

In an embodiment, the gene editing reagents are mixed with lipid nanoparticles. As used herein, the term “lipid nanoparticle” refers to a transfer vehicle comprising one or more lipids. The term “lipid nanoparticle” also refers to particles having at least one dimension on the order of nanometers (e.g., 1-1,000 nm) which include one or more of the compounds of formula (I) or other specified cationic lipids. The one or more lipids can be cationic lipids, non-cationic lipids, or PEG-modified lipids. The lipid nanoparticles can be formulated to deliver one or more gene editing reagents to one or more target cells. Examples of suitable lipids include phosphatidylglycerol, phosphatidylcholine, phosphatidylserine, phosphatidylethanolamine, sphingolipids, cerebrosides, and gangliosides). Also contemplated is the use of polymers as transfer vehicles, whether alone or in combination with other transfer vehicles. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, dendrimers and polyethylenimine. In one embodiment, the transfer vehicle is selected based upon its ability to facilitate the transfection of a gene editing reagent to a target cell.

In an embodiment, this document describes the use of lipid nanoparticles as transfer vehicles comprising a cationic lipid to encapsulate and/or enhance the delivery of a gene editing reagent into a target cell. As used herein, the phrase “cationic lipid” refers to any of a number of lipid species that carry a net positive charge at a selected pH, such as physiological pH. The contemplated lipid nanoparticles may be prepared by including multi-component lipid mixtures of varying ratios employing one or more cationic lipids, non-cationic lipids and PEG-modified lipids. In certain embodiments, the compositions and methods within this document employ lipid nanoparticles comprising (15Z,18Z)-N,N-dimethyl-6-(9Z,12Z)-octadeca-9,12-dien-1-yl)tetracosa-15,18-dien-1-amine (HGT5000), (15Z,18Z)-N,N-dimethyl-6-((9Z,12Z)-octadeca-9,12-dien-1-yl)tetracosa-4,15,18-trien-1-amine (HGT5001), or (15Z,18Z)-N,N-dimethyl-6-((9Z,12Z)-octadeca-9,12-dien-1-yl)tetracosa-5,15,18-trien-1-amine (HGT5002).

In an embodiment, the gene editing reagents can be delivered with the lipid nanoparticle BAMEA-016B. The gene editing reagents can be in the form of RNA. For example, the gene editing reagents can be Cas9 mRNA and sgRNA combined with BAMEA-016B lipid nanoparticles.

In certain embodiments, the cationic lipid N-[1-(2,3-dioleyloxy)propyl]-N,N,N-trimethylammonium chloride (DOTMA) can be used. DOTMA can be formulated alone or combined with the neutral lipid, dioleoylphosphatidyl-ethanolamine (DOPE) or other cationic or non-cationic lipids into a liposomal transfer vehicle or a lipid nanoparticle, and such liposomes can be used to enhance the delivery of nucleic acids into target cells. Other suitable cationic lipids include, 5-carboxyspermylglycinedioctadecylamide,” 2,3-dioleyloxy-N-[2(spermine-carboxamido)ethyl]-N,N-dimethyl-1-propanaminium, 1,2-Dioleoyl-3-Dimethylammonium-Propane, 1,2-Dioleoyl-3-Trimethylammonium-Propane. Contemplated cationic lipids also include 1,2-distearyloxy-N,N-dimethyl-3-aminopropane, 1,2-dioleyloxy-N,N-dimethyl-3-aminopropane, 1,2-dilinoleyloxy-N,N-dimethyl-3-aminopropane, 1,2-dilinolenyloxy-N,N-dimethyl-3-aminopropane, N-dioleyl-N,N-dimethylammonium chloride, N,N-distearyl-N,N-dimethylammonium bromide, N-(1,2-dimyristyloxyprop-3-yl)-N,N-dimethyl-N-hydroxyethyl ammonium bromide, 3-dimethylamino-2-(cholest-5-en-3-beta-oxybutan-4-oxy)-1-(cis,cis-9,12-octadecadienoxy)propane, 2-[5′-(cholest-5-en-3-beta-oxy)-3′-oxapentoxy)-3-dimethyl-1-(cis,cis-9′,1-2′-octadecadienoxy)propane, N,N-dimethyl-3,4-dioleyloxybenzylamine, 1,2-N,N′-dioleylcarbamyl-3-dimethylaminopropane, 2,3-Dilinoleoyloxy-N,N-dimethylpropylamine, 1,2-N,N′-Dilinoleylcarbamyl-3-dimethylaminopropane, 1,2-Dilinoleoylcarbamyl-3-dimethylaminopropane, 2,2-dilinoleyl-4-dimethylaminomethyl-[1,3]-dioxolane, 2,2-dilinoleyl-4-dimethylaminoethyl-[1,3]-dioxolane, and 2-(2,2-di((9Z,12Z)-octadeca-9,12-dien-1-yl)-1,3-dioxolan-4-yl)-N,N-dimethylethanamine (DLin-KC2-DMA)), or mixtures thereof.

In certain embodiments, cholesterol-based cationic lipids can be used to facilitate delivery of gene editing reagents to target cells in the present document. Cholesterol-based cationic lipids can be used alone or in combination with other cationic or non-cationic lipids. Suitable cholesterol-based cationic lipids include DC-Chol (N,N-dimethyl-N-ethylcarboxamidocholesterol), or 1,4-bis(3-N-oleylamino-propyl)piperazine.

In certain embodiments, cationic lipids such as the dialkylamino-based, imidazole-based, and guanidinium-based lipids are used to facilitate delivery of gene editing reagents to target cells in the present document. For example, certain embodiments are directed to a composition comprising one or more imidazole-based cationic lipids, for example, the imidazole cholesterol ester or “ICE” lipid (3S,10R,13R,17R)-10,13-dimethyl-17-(R)-6-methylheptan-2-yl)-2,3,4,7,8,9,10,11,12,13,14,15,16,17-tetradecahydro-1H-cyclopenta[a]phenanthren-3-yl 3-(1H-imidazol-4-yl)propanoate.

The imidazole-based cationic lipids are also characterized by their reduced toxicity relative to other cationic lipids. The imidazole-based cationic lipids (e.g., ICE) may be used as the sole cationic lipid in the lipid nanoparticle, or alternatively may be combined with traditional cationic lipids, non-cationic lipids, and PEG-modified lipids. The cationic lipid may comprise a molar ratio of about 1% to about 90%, about 2% to about 70%, about 5% to about 50%, about 10% to about 40% of the total lipid present in the transfer vehicle, or preferably about 20% to about 70% of the total lipid present in the transfer vehicle.

In other embodiments the gene editing reagents and methods described herein are use lipid nanoparticles comprising one or more cleavable lipids, such as, for example, one or more cationic lipids or compounds that comprise a cleavable disulfide (S—S) functional group (e.g., HGT4001, HGT4002, HGT4003, HGT4004 and HGT4005).

The use of polyethylene glycol (PEG)-modified phospholipids and derivatized lipids such as derivatized ceramides (PEG-CER), including N-Octanoyl-Sphingosine-1-[Succinyl(Methoxy Polyethylene Glycol)-2000] (C8 PEG-2000 ceramide) is also contemplated by the present invention, either alone or preferably in combination with other lipids together which comprise the transfer vehicle (e.g., a lipid nanoparticle). Contemplated PEG-modified lipids include, but is not limited to, a polyethylene glycol chain of up to 5 kDa in length covalently attached to a lipid with alkyl chain(s) of C6-C20 length. The addition of such components may prevent complex aggregation and may also provide a means for increasing circulation lifetime and increasing the delivery of the lipid-nucleic acid composition to the target cell, or they may be selected to rapidly exchange out of the formulation in vivo. Particularly useful exchangeable lipids are PEG-ceramides having shorter acyl chains (e.g., C14 or C18). The PEG-modified phospholipid and derivatized lipids of the present invention may comprise a molar ratio from about 0% to about 20%, about 0.5% to about 20%, about 1% to about 15%, about 4% to about 10%, or about 2% of the total lipid present in the liposomal transfer vehicle.

The present document also contemplates the use of non-cationic lipids. As used herein, the phrase “non-cationic lipid” refers to any neutral, zwitterionic or anionic lipid. As used herein, the phrase “anionic lipid” refers to any of a number of lipid species that carry a net negative charge at a selected pH, such as physiological pH. Non-cationic lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof. Such non-cationic lipids may be used alone, but are preferably used in combination with other excipients, for example, cationic lipids. When used in combination with a cationic lipid, the non-cationic lipid may comprise a molar ratio of 5% to about 90%, or preferably about 10% to about 70% of the total lipid present in the transfer vehicle.

In one embodiment, the lipid nanoparticle is prepared by combining multiple lipid and/or polymer components. For example, a transfer vehicle may be prepared using C12-200, DOPE, chol, DMG-PEG2K at a molar ratio of 40:30:25:5, or DODAP, DOPE, cholesterol, DMG-PEG2K at a molar ratio of 18:56:20:6, or HGT5000, DOPE, chol, DMG-PEG2K at a molar ratio of 40:20:35:5, or HGT5001, DOPE, chol, DMG-PEG2K at a molar ratio of 40:20:35:5. The selection of cationic lipids, non-cationic lipids and/or PEG-modified lipids which comprise the lipid nanoparticle, as well as the relative molar ratio of such lipids to each other, is based upon the characteristics of the selected lipid(s), the nature of the intended target cells, the characteristics of the mRNA to be delivered. Additional considerations include, for example, the saturation of the alkyl chain, as well as the size, charge, pH, pKa, fusogenicity and toxicity of the selected lipid(s). The molar ratios may be adjusted accordingly. For example, in embodiments, the percentage of cationic lipid in the lipid nanoparticle may be greater than 10%, greater than 20%, greater than 30%, greater than 40%, greater than 50%, greater than 60%, or greater than 70%. The percentage of non-cationic lipid in the lipid nanoparticle may be greater than 5%, greater than 10%, greater than 20%, greater than 30%, or greater than 40%. The percentage of cholesterol in the lipid nanoparticle may be greater than 10%, greater than 20%, greater than 30%, or greater than 40%. The percentage of PEG-modified lipid in the lipid nanoparticle may be greater than 1%, greater than 2%, greater than 5%, greater than 10%, or greater than 20%.

In certain embodiments, the lipid nanoparticles can comprise at least one of the following cationic lipids: C12-200, DLin-KC2-DMA, DODAP, HGT4003, ICE, HGT5000, or HGT5001. In embodiments, the transfer vehicle comprises cholesterol and/or a PEG-modified lipid. In some embodiments, the transfer vehicles comprise DMG-PEG2K. In certain embodiments, the transfer vehicle comprises one of the following lipid formulations: C12-200, DOPE, DMG-PEG2K; DODAP, DOPE, cholesterol, DMG-PEG2K; HGT5000, DOPE, DMG-PEG2K, HGT5001, DOPE, DMG-PEG2K.

The liposomal transfer vehicles for use with the gene editing reagents of the invention can be prepared by various techniques. For example, multi-lamellar vesicles (MLV) are prepared by depositing a selected lipid on the inside wall of a suitable container or vessel by dissolving the lipid in an appropriate solvent, and then evaporating the solvent to leave a thin film on the inside of the vessel or by spray drying. An aqueous phase may then be added to the vessel with a vortexing motion which results in the formation of MLVs. Uni-lamellar vesicles (ULV) can then be formed by homogenization, sonication or extrusion of the multi-lamellar vesicles. In addition, unilamellar vesicles can be formed by detergent removal techniques.

Liposomal transfer vehicles may be designed according to delivering gene editing reagents to target organs. For example, to target hepatocytes in the liver, a liposomal transfer vehicle may be sized such that its dimensions are smaller than the fenestrations of the endothelial layer lining within the liver. In various embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic.

As described herein, a gene editing reagent (e.g., a nuclease in mRNA format) may be mixed with lipid nanoparticles and delivered locally to a target organ. Local delivery can refer to delivery of lipid nanoparticles with gene editing reagents in a lumen in proximity to or within an organ-of-interest. The local delivery can be achieved through medical devices, such as catheters.

In other embodiments, this document provides GECs with customized accessories that facilitate the depositing of reagent into organs. One accessory is the needle (FIG. 1). When located at an intra-organ position, a GEC outfitted with a needle can penetrate the lumen wall and release the gene editing reagents directly into the organ. The GEC can be customized with 1, 2, 3, 4, 5, 6, 7, 8, 9, 10 or more needles. The needles can be positioned around the circumference of the GEC, or the needles can be positioned down the length of the GEC. Needles can have a gauge of between 15 and 34. The needle can have a gauge of 15, 16, 17, 18, 19, 20, 21, 22, 23, 24, 25, 26, 27, 28, 29, 30, 31, 32, 33, or 34. The needles can be microneedles and the gene editing solution can be delivered through channels within the needles or the pores created by the microneedles.

In other embodiments, this document provides GECs that can be used in conjunction with accessories that facilitate the uptake of the gene editing reagent into organ cells. In certain embodiments, these accessories can be customized to be associated directly with the catheter (i.e., integrated into catheter design). One exemplary accessory is the electrode (FIG. 1). When located at the proximity or intra-organ position, a GEC with an electrode can introduce an electrical pulse through the organ-of-interest. The electrical pulse can result in the pores of cell membranes briefly opening to allow the gene editing reagent to enter. Both exponential-decay and square-wave pulses can be used for electroporation. The field intensity can be between about 1 and 600 Volts, between 1 and 400 Volts, between about 1 and 200 Volts, between about 10 and 100 Volts, or between 15 and 70 Volts. The total duration of application of the electric field may be between 0.01 millisecond and 1 second, between 0.01 and 500 milliseconds, or between 1 and 500 milliseconds. In one embodiment, the total duration of application of the electric field is 20 milliseconds. The number of electric pulses applied may be between, for example, 1 and 100,000. Their frequency may be between 0.1 and 1,000 Hertz. Electric pulses may also be delivered in an irregular manner relative to each other, the function describing the intensity of the electric field as a function of the time for one pulse being preferably variable. Electric pulses may be unipolar or bipolar wave pulses. They may be selected for example from square wave pulses, exponentially decreasing wave pulses, oscillating unipolar wave pulses of limited duration, oscillating bipolar wave pulses of limited duration, or other wave forms. Electric pulses comprise square wave pulses or oscillating bipolar wave pulses. To increase the number of transfected cells within the target organ, multiple rounds of the gene editing reagent can be deposited followed by multiple pulses of electricity.

In some embodiments, other exemplary accessories include the use of one or more magnets (FIG. 1). To facilitate cellular uptake, in certain embodiments the gene editing reagent is attached to magnetic nanoparticles. A magnetic field generated by the GEC can facilitate the concentration or movement of the gene editing reagents onto the cells, which is followed by cellular uptake through endocytosis and pinocytosis. In other embodiments, an external magnetic field is applied to the target organ to facilitate uptake of the gene editing reagents.

In some embodiments, the magnet can be a permanent magnet or an electromagnet. The permanent magnet can be comprised of neodymium iron boron (NdFeB), samarium cobalt (SmCo), alnico (aluminum, nickel and cobalt), or ceramic or ferrite. The electromagnet can comprise a wire wound around a magnetic or non-magnetic core. The core can contain nickel, cobalt, iron, steel, neodymium non-magnetic material, or ferro-magnetic metals.

The strength of the magnetic field produced by the permanent magnet or electromagnet can be between 100 micro tesla (μT) and 10 T, between 1 mT and 2 T, or between 100 mT and 0.5 T. Following or during depositing of the gene editing reagents a magnetic field can be applied to the target organ. The duration that the magnetic field is applied can be between 0.1 milliseconds and 1 hour, between 1 second and 10 minutes between 1 minute and 10 minutes, between 5 minutes and 10 minutes. In one embodiment, during the duration that the magnetic field is applied, the magnetic field can be reversed. In another embodiment, during the duration that the magnetic field is applied, the magnetic field can be pulsed or oscillated. The frequency of the pulse can be between 1000 hertz (Hz) and 0.01 Hz. The duration of the pulse can be between 1 millisecond second and 1 minute.

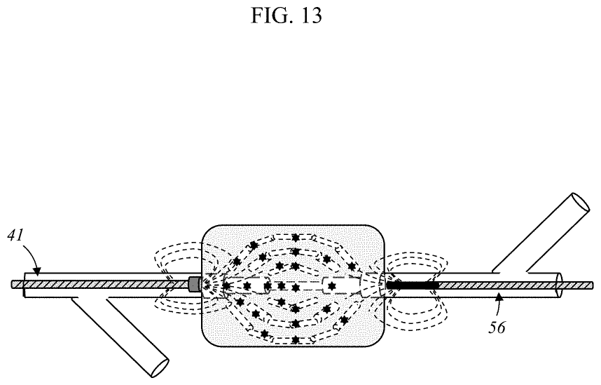

In one embodiment, a GEC can harbor a permanent magnet or electromagnet. The magnetic field can be applied before, during or after depositing gene editing reagents. In another embodiment, a permanent magnet or electromagnet can be harbored within a GEC and within a second catheter positioned in a lumen carrying fluid away from the organ (FIG. 13). In another embodiment, a permanent magnet or electromagnet can be harbored within a second catheter positioned in a lumen carrying fluid away from the organ.

In one embodiment, gene editing reagents dispensed by the GEC can be bound to magnetic nanoparticles. The magnetic nanoparticle can be an iron oxide, including magnetite (Fe₃O₄) or maghemite (γ-Fe₂O₃). The magnetic nanoparticle can be CoFe₂O₄, NiFe₂O₄, or MnFe₂O₄. The magnetic nanoparticle can be coated with an agent to prevent agglomeration, cytotoxicity or to add functionality. The coating can be a natural polymer (protein or carbohydrate), synthetic organic polymers (polyethylene glycol), polyvinyl alcohol, poly-1-lactic acid), silica, or gold. The coating can be anionic surfactants (oleic acid, lauroyl sarcosinate), a non-ionic water-soluble surfactant (Pluronic F-127), fluorinated surfactant (lithium 3-[2-(perfluoroalkyl) ethylthio]propionate), a polymer (polyethylene glycol, poly-1-lysine, poly(propyleneimine) dendrimers), carbohydrates (Chitosan, Heparan sulfate), silica particles (MCM48), proteins (serum albumin, streptavidin), hydroxyapatite, phospholipids, a cationic cell penetrating peptide (TAT peptide), non-activated virus envelope (HVJ-E), a transfection reagent (Lipofectamine 2000), and viruses (adenovirus, retrovirus). The coating agents can be used in conjugation with polyethylenimine (PEI). The size of the nanoparticle delivered by the GEC can be between 1 nanometer and 1 micrometer, or between 10 nanometers and 200 nanometers. The gene editing reagents can be bound to the magnetic nanoparticles and can be in the form of protein, RNA or DNA.

In some embodiments, the magnetic nanoparticle can include particles with added features. For example, the magnetic nanoparticle can be a magnetic micro propeller (Schuerle et al., Science Advances, 5(4), eaav4803, 2019) or a microrobot in the shape of a cylinder, hexahedral, helix, or sphere (e.g., Jeon et al., Science Robotics, 4(30), eaav4317, 2019).

In some embodiments, the magnetic field is applied by an external device. Gene editing reagents bound to magnetic nanoparticles can be delivered to a target organ followed by, or simultaneously to, exposure of the target organ to a magnetic field by an external device. The external device can be a permanent or electromagnet which is placed nearby or adjacent to the target organ. Alternatively, the magnetic field can be produced by a magnetic resonance imaging (Mill) system. The external device can produce a magnetic field of between 100 micro tesla (μT) and 10 T, between 1 mT and 2 T, or between 100 mT and 0.5 T. The duration of the magnetic field can be between 0.1 milliseconds and 1 hour, between 1 second and 10 minutes between 1 minute and 10 minutes, between 5 minutes and 10 minutes. In one embodiment, during the duration that the magnetic field is applied, the magnetic field can be reversed. In another embodiment, during the duration that the magnetic field is applied, the magnetic field can be pulsed or oscillated. The frequency of the pulse can be between 1000 hertz (Hz) and 0.01 Hz. The duration of the pulse can be between 1 millisecond second and 1 minute.

In some embodiments, the devices described within this document can be used to deliver cells with one or more gene edits, where the cells were edited in vitro or ex vivo by one or more gene editing reagents.

In some embodiments, other exemplary accessories include a sonicator (FIG. 1). To facilitate cellular uptake, gene editing reagents are associated with gas-encapsulated microbubbles and deposited in proximity or intra-organ. An acoustic field generated by the GEC creates oscillations which cause fragmentation of the microbubble, resulting in a momentum transfer which induces poration of the cell membrane and induction of endocytosis.

In certain embodiments, the catheter assembly comprising the reagent dispenser, permanent magnet, electromagnet, needle, sonicator or electrode can comprise flexible tubing. The flexible tubing can be silicone rubber, nylon, polyurethane, polyethylene terephthalate (PET), latex, vinyl, thermoplastic elastomers, multilayer tubing, polyimide tubing, polytetrafluoroethylene, liner tubing, or reinforced tubing. The catheter tubing or shaft can have a diameter sufficient for delivering liquids or gels to a target organ. The flexible tubing can have an outer diameter of 0.90 inches or less, 0.30 inches or less, 0.20 inches or less, 0.15 inches or less, 0.10 inches or less, 0.08 inches or less, 0.06 inches or less, 0.05 inches or less, or 0.04 inches or less. The catheter can have one or more channels. The catheter can be a single channel catheter with the permanent magnet or electromagnet housed within or outside of the channel. The catheter can be dual-channel, triple channel or quadruple channel. The catheter can comprise combinations of the permanent magnet, electromagnet, needle, sonicator or electrode. The catheter can comprise an electromagnet and sonicator, an electromagnet and needle, an electromagnet and electrode, a needle and sonicator, a needle and electrode, or a sonicator and electrode.

The catheter assembly comprising the reagent dispenser, permanent magnet, electromagnet, needle, sonicator or electrode can also comprise a guide wire. The guide wire can be solid steel or nitinol, or solid core wire wrapped in a smaller wire coil or braid. The guide wire can be coated with a polymer, including silicone or polytetrafluoroethylene. Guide wire diameter can be between 0.014 and 0.038 inches.

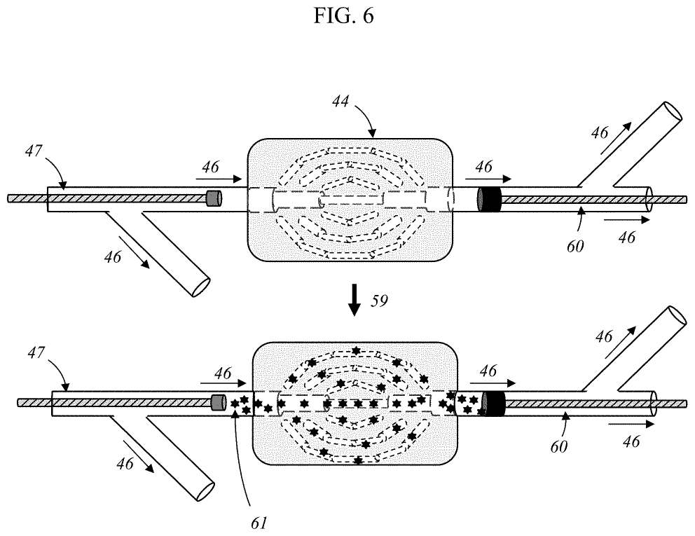

In another embodiment, this document provides methods for reducing or eliminating the systemic spread of gene editing reagents to non-target tissues/organs (FIG. 6). The method can include the use of catheters which can be inserted into lumens, including blood vessels, ducts, or the gastrointestinal tract. The catheters described herein are customized and tailored to capture or inactivate gene editing reagents exiting a target organ. Further, the gene editing reagents can be customized and tailored to facilitate capture or inactivation by the catheter.

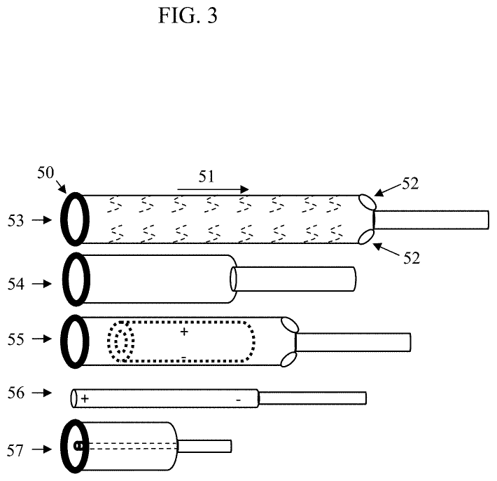

The catheter that captures or inactivates gene editing reagents exiting a target organ is herein referred to as the safety gene editing catheter (S-GEC). An S-GEC can comprise medical grade materials which form a flexible, thin tube. The S-GEC can be inserted through openings or lumens within the subject. The S-GEC can comprise a device which creates a seal between the catheter circumference and the lumen wall, resulting in all or most of the lumen fluid being directed through the S-GEC (FIG. 3). In one embodiment, the device can comprise a balloon that runs along the circumference of the distal end of the catheter. When positioned within a lumen, the inflation of the balloon creates a seal between the catheter and lumen wall. The S-GEC can be outfitted with one or more devices which facilitate the capture or inactivate the gene editing reagents (FIG. 3).

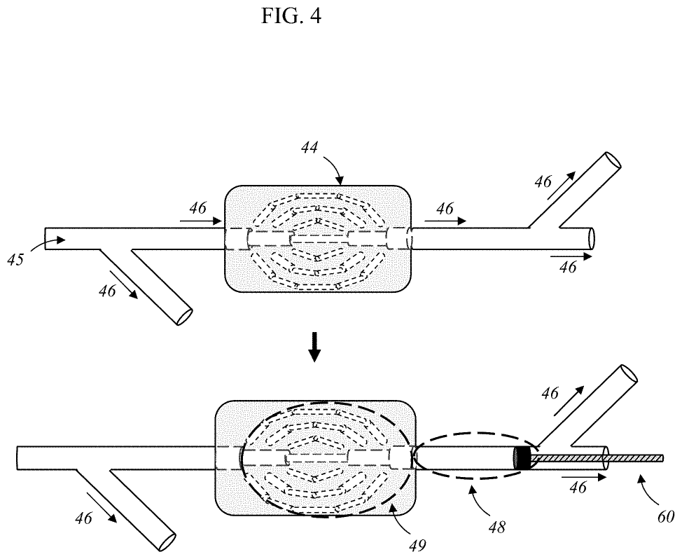

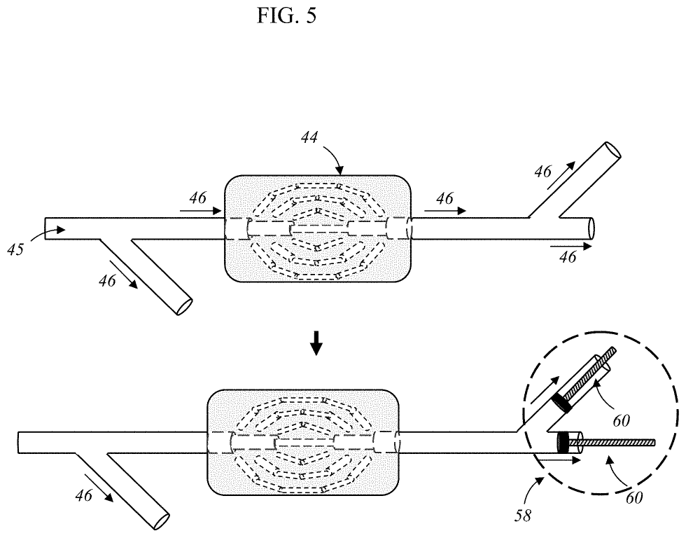

In another embodiment, this document provides methods for positioning the S-GEC within the subject. To capture of gene editing reagents exiting an organ-of-interest, the S-GEC can be positioned within lumens comprising fluid exiting the organ-of-interest. The S-GEC can be positioned in one or more lumens exiting the organ-of-interest. The S-GEC can be positioned in a lumen where branching following the organ-of-interest has not yet occurred (FIG. 4). Alternatively, the S-GEC can be positioned in lumens following branching (FIG. 5).

The capture of gene editing reagents by the S-GEC can be achieved using multiple mechanisms, including the use of (i) collection tubes which diverts fluid leaving the organ to a collection apparatus or dialysis machine, (ii) binding elements such as antibodies or glutathione-coated plates which sequester gene editing reagents, (iii) charged elements such as magnets which capture nucleic acids or virus particles bound to magnetic beads, (iv) size exclusion elements. To inactivate gene editing reagents, the S-GEC can comprise a reagent dispenser. The reagent dispenser can deposit proteins or molecules which inactivate or destroy gene editing reagents. The protein can include a restriction endonuclease, DNase, RNase, an RNA oligonucleotide inhibitor (Barkau et al, Nucleic Acid Ther 29:136-147, 2019), or anti-CRISPR protein. The reagent dispenser can deposit the proteins or molecules into the lumen following the organ-of-interest at the same time or shortly following the time the gene editing reagents are delivered.

In one embodiment, this document provides S-GECs that capture gene editing reagents using a purification system comprising immobilized proteins, small peptides, chemicals, or nucleic acids that bind to, sequester, or inactivate the gene editing reagent. The immobilized proteins, small peptides, chemicals, or nucleic acids can be an antibody that recognizes and binds to the gene editing reagent. The immobilized protein, small peptides or nucleic acids can be an anti-CRISPR peptide that binds to the gene editing reagent. The immobilized proteins, small peptides, chemicals, or nucleic acids can be glutathione which binds to a glutathione S-transferase (GST) tag present on the gene editing reagent. The immobilized proteins, small peptides, chemicals, or nucleic acids can be a protease or nuclease that destroys the gene editing reagent as it passes through the S-GEC.

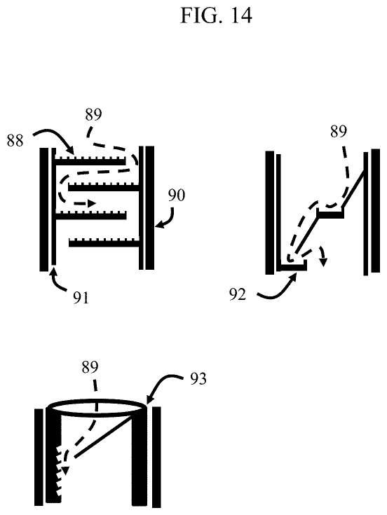

In another embodiment, this document provides S-GECs that capture gene editing reagents using a purification system with charged substrates or material. The charged substrate or material can capture gene editing reagents with the opposite charge. In one embodiment, the GEC delivers gene editing reagents in the form of nucleic acid or protein bound to cationic metal beads or magnetic nanoparticles. To capture gene editing reagents, the S-GEC can comprise a diametrically magnetized ring. Fluid passing through the magnetized ring will be subject to the magnetic field. Illustrations of different S-GEC designs for capturing magnetic nanoparticles is shown in FIG. 14.

In another embodiment, the charged substrate can be a cylindrical magnet with poles on opposite ends of the cylinder. The negative pole can be facing towards the organ-of-interest. Magnetic beads within the organ-of-interest will be pulled through organ and captured by the negative pole. Alternatively, the positive pole can be facing towards the organ-of-interest. Magnetic beads within the organ-of-interest will be pushed away from the vein(s) exiting the organ. Magnetic beads which both exit the organ and pass by the positive pole can be captured by the negative pole.

In other embodiments, this document provides S-GECs with customized accessories that facilitate the uptake of the gene editing reagent into organ cells. One accessory is the electrode (FIG. 3). When located at the proximity, adjacent or intra-organ positions, an S-GEC with an electrode can facilitate the transmission of an electrical pulse through the organ-of-interest. The electrical pulse can result in the pores of cell membranes briefly opening to allow the gene editing reagent to enter.

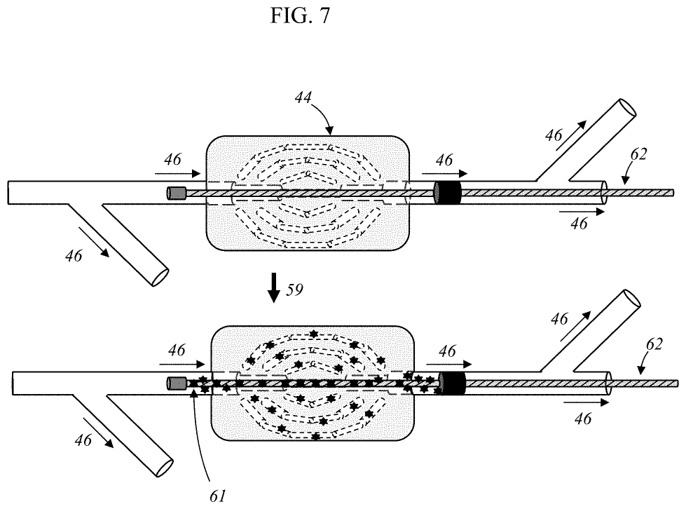

In one embodiment, this document provides devices which comprise both a GEC and S-GEC in a single component system. This single component system is herein referred to as a SS-GEC (FIG. 7). The SS-GEC can be used to both deliver the gene editing reagent to a target organ and capture the gene editing reagents leaving the organ. Here, the SS-GEC traverses the organ-of-interest to position the dispenser for the gene editing reagent. The dispenser can be positioned in a lumen where the fluid is flowing into the organ-of-interest. The device that captures or inactivates the gene editing reagents can be positioned within the lumen where fluids are flowing out of the organ-of-interest.

The SS-GEC can be customized with accessories that facilitate the uptake of the gene editing reagents into organ cells. The accessories include needles, electrodes, magnets or sonicators. To increase the number of cells within the organ that are correctly edited, the SS-GEC can be used to administer multiple rounds of gene editing reagents.

The devices described in this document can be used together with gene editing reagents, including CRISPR, TALENs, ZFNs and donor molecules. The CRISPR system can include CRISPR/Cas9 or CRISPR/Cpfl. The CRISPR system can include the use of variants which display broad PAM capability (Hu et al., Nature 556, 57-63, 2018) or higher on-target binding or cleavage activity (Kleinstiver et al., Nature 529:490-495, 2016). The gene editing reagent can be in the format of a nuclease (Mali et al., Science 339:823-826, 2013; Christian et al., Genetics 186:757-761, 2010), nickase (Cong et al., Science 339:819-823, 2013; Wu et al., Biochemical and Biophysical Research Communications 1:261-266, 2014), base editors (Komor et al., Nature 533:420-424, 2016), RNA editors (Cox et al., Science 358:1019-1027, 2017), CRISPR-FokI dimers (Tsai et al., Nature Biotechnology 32:569-576, 2014), paired CRISPR nickases (Ran et al., Cell 154:1380-1389, 2013), TALE activator (Maeder et al., Nature Methods 10:243-245, 2013), TALE repressor (Cong et al., Nature Communications 3:968, 2012), CRISPR activator (Cheng et al., Cell Research 23:1163-1171, 2013), or CRISPR repressor (Qi et al., Cell 152:1173-1183, 2013; Thakore et al., Nature Methods 12:1143-1149, 2015).

The capture or inactivation of a gene editing reagent by an S-GEC can be facilitated by the inherent properties or intentional design of the gene editing reagent. Inherent properties that facilitate inactivation include anti-CRISPR proteins. Intentionally designed properties include the addition of purification tags on the N or C terminus of CRISPR, TALEN or ZFN proteins. The tag can include a chitin binding protein (CBP) tag, maltose binding protein (MBP) tag, Strep-tag, FLAG tag, or glutathione-S-transferase (GST) tag.

In one embodiment, the methods provided in this document can be used for the delivery of gene editing reagents that facilitate the destruction of cells within a target organ. In a specific embodiment, destruction can be facilitated with the use of CRISPR systems which, following targeted cleavage of a target RNA, exhibit collateral RNase activity. The CRISPR system can include the Class 2 subtype VI-B Cas13b system (Smargon et al., Molecular Cell 65:618-630, 2017). In other embodiments, destruction can be facilitated by CRISPR/Cas9 or Cpfl, TALENs or ZFNs which target a sufficient number of genomic sequences within a cell or target genes essential for survival.

The methods described herein for the destruction of cells can be used to reduce or eliminate the growth of cancer cells within organs. In one embodiment, cancer cells within the prostate are targeted for destruction. Here, a GEC is positioned within the urethra immediately adjacent to the prostate. The GEC can comprise needles which penetrate the urethra wall and enter the prostate. Gene editing reagents are then deposited within the prostate. Alternatively, the GEC is positioned within the left or right prostatic arteries, followed by the release of the gene editing reagents. The gene editing reagents can comprise the Cas13b, Cas9 or Cpfl systems, wherein expression of the Cas13b, Cas9 or Cpfl systems results in cell death.

In other embodiments, gene editing reagents which induce cell death can be delivered to tumors. The GEC can be positioned in one or more arteries supplying oxygen to tumor cells. In other embodiments, an S-GEC can be positioned in one or more veins carrying blood away from the tumor.

Also provided herein is a kit, which may be used to deliver genome editing reagents to organs in a human or animal. In one embodiment, the kit comprises a solution comprising at least one gene editing reagent, a catheter and instructions for using said catheter and solution. Instructions included in kits may be affixed to packaging material or may be included as a package insert. In certain embodiments, the instructions will direct a user to (i) insert the catheter (e.g., distal catheter end) into a lumen in proximity to or within a target organ; (ii) deliver a genome editing reagent to the organ through the catheter (e.g., via introduction of reagent through proximal end of catheter); and, optionally, (iii) activating an accessory to effect uptake of the at least one gene editing reagent by the organ cells. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions. In another embodiment, the kit comprises a solution comprising at least one gene editing reagent, a first and second catheter, and instructions for using said first and second catheters and solution. In another embodiment, the kit comprises a solution comprising at least one gene editing reagent, a catheter, a guidewire and instructions for using said catheter, said guidewire and solution. In another embodiment, the kit comprises a solution comprising at least one gene editing reagent, a first and second catheter, a guide wire, and instructions for using said first and second catheters and solution. In another embodiment, the kit comprises a solution comprising at least one gene editing reagent, a first and second catheter, a guidewire, and instructions for using said first and second catheters, said guidewire and solution. In another embodiment, the kit comprises a first solution comprising at least one gene editing reagent, a first and second catheter, a second solution comprising at least one component to inactivate or destroy said gene editing reagent, and instructions for using said first and second catheters with said first and second solutions. In another embodiment, the kit comprises a first solution comprising at least one gene editing reagent, a first and second catheter, a second solution comprising at least one component to inactivate or destroy said gene editing reagent, a guidewire and instructions for using said first and second catheters with said first and second solutions and guidewire.

The devices and methods described in this document can be used to edit the genome of organ cells in vivo. The organ can be the liver, kidneys, bladder, muscular system, pharynx, esophagus, stomach, small intestine, duodenum, jejunum, ileum, large intestine, gallbladder, mesentery, pancreas, nasal cavity, pharynx, larynx, trachea, bronchi, lungs, diaphragm, ureters, urethra, ovaries, fallopian tubes, uterus, testes, epididymis, vas deferens, seminal vesicles, prostate, bulbourethral glands, penis, endocrine system, pituitary gland, pineal gland, thyroid gland, parathyroid glands, adrenal glands, pancreas, heart, lymph node, bone marrow, thymus, spleen, tonsils, nervous system, brain, cerebrum, cerebral hemispheres, diencephalon, the brainstem, midbrain, pons, medulla oblongata, cerebellum, the spinal cord, the ventricular system, choroid plexus, skin, or subcutaneous tissue.

The devices and methods described in this document can be used to deliver the gene editing reagents for modifying cells of interest within an organ-of-interest. The organ can be the pancreas and the target cells can be cells within islets of Langerhans.

In one embodiment, the methods for preventing systemic spread of gene editing reagents can include the removal of lymph fluid before entering back into the blood stream. In another embodiment, the methods can include the use of an S-GEC within the lymphatic system to capture or inactivate gene editing reagents.

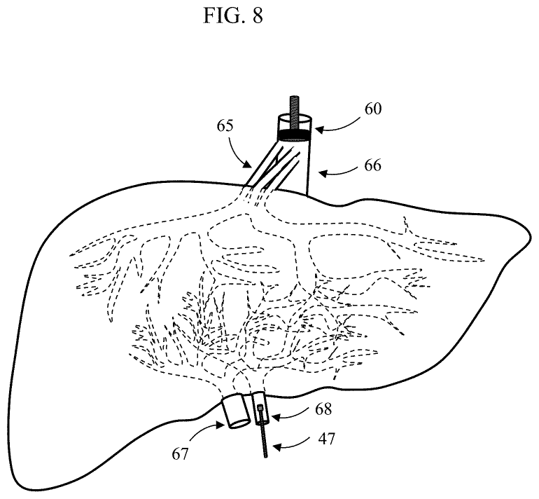

In some embodiments, the methods provided herein can be used in a human, mouse, rat, guinea pig, hamster, dog, pig, sheep, chimpanzee, monkey, horse or cow. The devices and methods provided herein can be used for the modification of liver cells. The methods include the use of GECs for localized administration of the gene editing reagent with or without the use of S-GECs for reducing or preventing the systemic spread of gene editing reagents throughout the subject. To deliver gene editing reagents to the liver, the GEC can be positioned at several different sites. The first site includes the hepatic artery proper (FIG. 8). A second site includes the right and left hepatic arteries. A third site includes the branches of the hepatic artery. A fourth site includes the portal vein. A fifth site includes the branches of the portal vein.

Gene editing reagents exiting the liver can be captured with the use of S-GECs. To capture gene editing reagents, an S-GEC can be positioned within the hepatic veins, including the right hepatic vein, left hepatic vein and middle hepatic vein. Alternatively, the S-GEC can be positioned within the inferior vena cava following the connection sites of the hepatic veins (FIG. 8).

Instead of using GEC and S-GEC devices for the delivery and capture of gene editing reagents in the liver, a single component SS-GEC can be used. In one embodiment, the SS-GEC device comprises both a reagent dispenser followed by a device that captures or inactivates the gene editing reagent.

The methods and materials described herein can be used in the liver for the treatment of conditions such as Crigler-Najjar syndrome type 1 (CN1), familial hypercholesterolemia and other lipid metabolic disorders, maple syrup urine disease, progressive familial intrahepatic cholestasis, phenylketonuria, tyrosinemia, mucopolysaccharidosis VII, AAT deficiency, OTC deficiency, Wilson's disease, glycogen storage diseases (e.g., von Gierke's disease and Pompe's disease), hyperbilirubinema, acute intermittent porphyria, citrullinemia type 1, hemophilia A and B, oxalosis, infectious diseases (e.g., hepatitis B and C), malignant neoplasms (hepatomas, cholangiocarcinomas, and metastatic tumors), extrahepatic tumors (inhibition of neovascularization), cirrhosis of the liver, allograft or xenograft rejection.

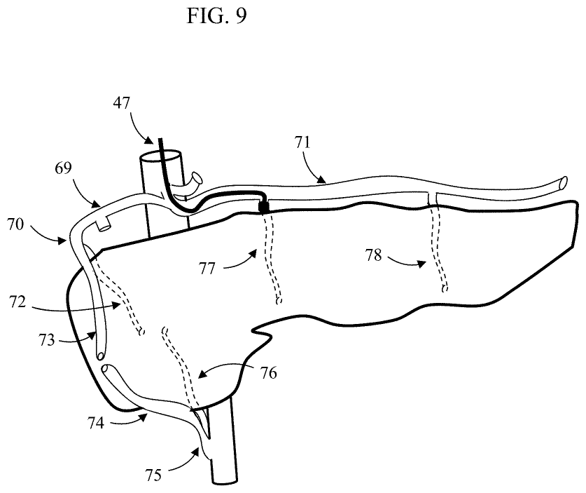

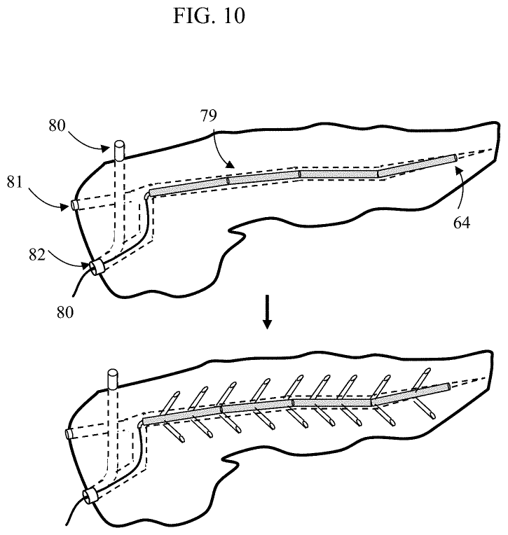

The devices and methods provided herein can be used for the modification of pancreas cells. The methods include the use of GECs for localized administration of reagent with or without the use of S-GECs for reducing or preventing the systemic spread of gene editing reagents throughout the subject. To deliver gene editing reagents to the pancreas, the GEC can be positioned at several different sites. The first site includes the intra-organ delivery within the pancreatic ducts (FIG. 10). A second site includes the greater pancreatic artery. The third site includes dorsal pancreatic artery (FIG. 9). A fourth site includes the superior pancreatic duodenal artery. A fifth site includes the anterior superior pancreatic duodenal artery. A sixth site includes the posterior superior pancreatic duodenal artery. A seventh site includes the inferior pancreatic duodenal artery. An eighth site includes anterior superior pancreatic duodenal artery. A ninth site includes the inferior superior pancreatic duodenal artery.

Gene editing reagents exiting the pancreas through veins can be captured with the use of S-GECs. To capture gene editing reagents, an S-GEC can be positioned within the portal vein.

In another embodiment, this document provides methods for facilitating the entry of the genome editing reagents into pancreatic cells. Facilitation can occur through several mechanisms, including electroporation, nanoparticles, viruses, or disruption of the artery, vein or duct walls. In one example, a GEC can be positioned within the pancreatic duct. The GEC can comprise needles capable of penetrating the pancreatic duct walls. After penetration, the gene editing reagents can be dispensed. Following dispense of the gene editing reagents, an electrical pulse can be administered to the organ. Multiple rounds of reagent dispensing followed by electrical pulses can be used. In a second example, the GEC can deposit gene editing regents within the dorsal pancreatic artery. The devices and methods provided herein can be used for the modification of brain cells. The methods include the use of GECs for localized administration of reagent with or without the use of S-GECs for reducing or preventing the systemic spread of gene editing reagents throughout the subject. To deliver gene editing reagents to the brain, the GEC can be positioned at several different sites. The first site includes the left internal carotid artery. The second site includes the branches of the left internal carotid artery. The third site includes the right internal carotid artery. The fourth site includes the branches of the right internal carotid artery. The fifth site includes the left vertebral artery. The sixth site includes the branches of the left vertebral artery. The seventh site includes the right vertebral artery. The eighth site includes the branches of the right vertebral artery.

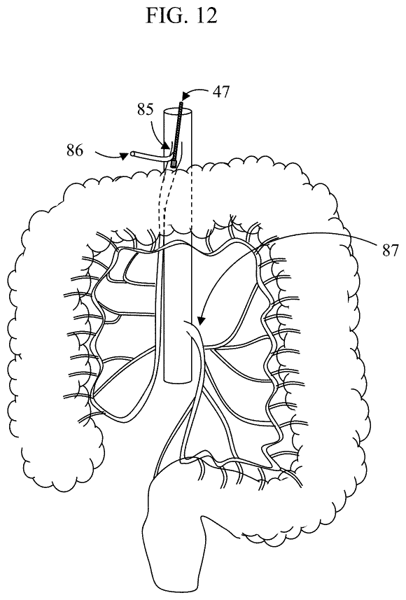

Gene editing reagents exiting the brain can be captured with the use of S-GECs. To capture gene editing reagents, two S-GECs can be positioned within the right and left internal jugular veins. The devices and methods provided herein can be used for the modification of cells within the gastrointestinal tract. The methods include the use of GECs for localized administration of reagent with or without the use of S-GECs for reducing or preventing the systemic spread of gene editing reagents throughout the subject. To deliver gene editing reagents to the gastrointestinal tract, the GEC can be positioned at several different sites. The first site includes the superior mesenteric artery following the inferior pancreaticoduodenal artery (FIG. 12). The second site includes the inferior mesenteric artery.

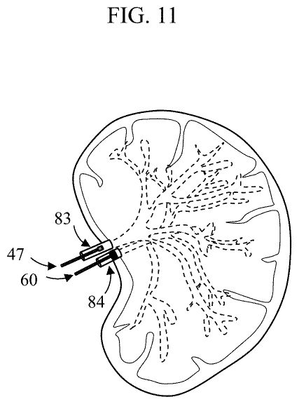

Gene editing reagents exiting the gastrointestinal tract can be captured with the use of S-GECs. To capture gene editing reagents, an S-GEC can be positioned within the portal vein. The devices and methods provided herein can be used for the modification of cells within the spleen. The methods include the use of GECs for localized administration of reagent with or without the use of S-GECs for reducing or preventing the systemic spread of gene editing reagents throughout the subject. To deliver gene editing reagents to the spleen, the GEC can be positioned at the splenic artery (FIG. 11). Gene editing reagents exiting the spleen can be captured with the use of S-GECs. To capture gene editing reagents, an S-GEC can be positioned within the splenic vein.

The devices and methods provided herein can be used for the modification of cells within the kidneys. The methods include the use of GECs for localized administration of reagent with or without the use of S-GECs for reducing or preventing the systemic spread of gene editing reagents throughout the subject. To deliver gene editing reagents to the kidneys, the GEC can be positioned at several different sites. The first site includes the renal artery. The second site includes the branches of the renal artery. The branches of the renal artery can include the anterior branch, inferior segmental, superior segmental, or posterior branch.

Gene editing reagents exiting the kidneys can be captured with the use of S-GECs. To capture gene editing reagents, an S-GEC can be positioned within the renal vein or within the ureter.

The devices and methods provided herein can be used for the modification of cells within the eye. The methods include the use of GECs for localized administration of reagent with or without the use of S-GECs for reducing or preventing the systemic spread of gene editing reagents throughout the subject. To deliver gene editing reagents to the eyes, including the retina and muscles of the eye, the GEC can be positioned at several different sites. The first site includes the central retinal artery. The second site includes the muscle branch of the ophthalmic artery. The third site includes the posterior ciliary artery.

Gene editing reagents exiting the eye can be captured with the use of S-GECs. To capture gene editing reagents, an S-GEC can be positioned within the superior ophthalmic vein, inferior ophthalmic vein, or cavernous sinus.

The devices and methods provided herein can be used for the editing or destruction of cells within the prostate. The methods include the use of GECs for localized administration of gene editing reagents within the prostate. To deliver gene editing reagents to prostate cells, the GEC can be positioned at multiple different sites. The first site includes the urethra at the point where the prostate surrounds the urethra. The second site includes the left or right inferior vesicle arteries. The third site includes the left or right prostatic arteries. The fourth site includes the branches of the left or right prostatic arteries.

When positioned within the urethra, the GEC can be customized with needles which penetrate the urethra wall, permitting the depositing of gene editing reagents within the prostate.

### Ex Vivo Delivery of Gene Editing Reagents

In other embodiments, this document provides methods for the delivery of gene editing reagents to cells within organs using ex vivo perfusion systems. As used herein, ex vivo perfusion of organs refers to techniques or procedures for maintaining organ viability within or outside a host. The methods described herein include pumping a perfusate or medical fluid through an organ and delivering one or more gene editing reagents. The organ can be subjected to normothermic perfusion, hypothermic perfusion, or perfusion at room temperature.

As referred to herein, the term “perfusate” or “medical fluid” refers to a fluid used in perfusion. The medical solution can be, for example, Belzer's Gluconate-Albumin Solution, University of Wisconsin Solution, histidine-tryptophan-ketoglutarate solution, blood, Lifor, or AQIX-RS-I. In some embodiments, the solution can further comprise an oxygen carrier, including perfluorocarbon and hemoglobin-based oxygen carriers.

The perfusion system can comprise a pump (e.g., a peristaltic pump or centrifugal pump) and one of several components. In some embodiments, the perfusion system can comprise one or more, or a combination of, a flow sensor, a blood/gas analyzing sensor, a flow sensor, a pressure sensor, a reservoir for medical fluid, oxygenator, gas filter, gas blender, heat exchanger, optical sensors, a chamber to hold the organ(s) or a dialysis machine.

In some embodiments, an organ can be removed from a host and connected to the perfusion system by attaching one or more arterial inlets to one or more tubes within the perfusion system, and one or more venous outlets to one or more tubes within the perfusion system. Medical fluid and gene editing solution can then be perfused through the organ. In one embodiment, the gene editing solution can be perfused alone. In other embodiments, the medical fluid can be administered before, during or after the administration of the gene editing solution. The gene editing solution may be administered through an arterial inlet (i.e., within tubing connected to arterial vasculature of the organ). In another embodiment, the gene editing solution may be delivered directly to the organ (e.g., using syringes to directly inject the gene editing solution). In other embodiments, after the gene editing solution is delivered, an external stimulus may be applied to the organ. The stimulus may be electricity, a magnetic field, or sonication.

In some embodiments, an organ can be maintained within the host and connected to a perfusion system by attaching one or more arterial inlets to one or more tubes within the perfusion system, and one or more venous outlets to one or more tubes within the perfusion system. Medical fluid and gene editing solution can then be perfused through the organ. The methods and materials described herein can be used to facilitate (i) the correction of a defective endogenous gene through gene targeting or base editors, (ii) the integration of a transgene or genetic element, (iii) the inactivation of an endogenous gene, (iv) the upregulation or downregulation of an endogenous gene, or (v) the destruction of a cell.

The transgene or endogenous gene can be a gene that is associated with a genetic disorder, including but not limited: achondroplasia, achromatopsia, acid maltase deficiency, adenosine deaminase deficiency (OMIM No. 102700), adrenoleukodystrophy, aicardi syndrome, alpha-1 antitrypsin deficiency, alpha-thalassemia, androgen insensitivity syndrome, apert syndrome, arrhythmogenic right ventricular, dysplasia, ataxia telangictasia, barth syndrome, beta-thalassemia, blue rubber bleb nevus syndrome, canavan disease, chronic granulomatous diseases (CGD), cri du chat syndrome, cystic fibrosis, dercum's disease, ectodermal dysplasia, fanconi anemia, fibrodysplasiaossificans progressive, fragile X syndrome, galactosemis, Gaucher's disease, generalized gangliosidoses (e.g., GM1), hemochromatosis, the hemoglobin C mutation in the 6th codon of beta-globin (HbC), hemophilia, Huntington's disease, Hurler Syndrome, hypophosphatasia, Klinefleter syndrome, Krabbes Disease, Langer-Giedion Syndrome, leukocyte adhesion deficiency (LAD, OMIM No. 116920), leukodystrophy, long QT syndrome, Marfan syndrome, Moebius syndrome, mucopolysaccharidosis (MPS), nail patella syndrome, nephrogenic diabetes insipdius, neurofibromatosis, Neimann-Pick disease, osteogenesisimperfecta, porphyria, Prader-Willi syndrome, progeria, Proteus syndrome, retinoblastoma, Rett syndrome, Rubinstein-Taybi syndrome, Sanfilippo syndrome, severe combined immunodeficiency (SCID), Shwachman syndrome, sickle cell disease (sickle cell anemia), Smith-Magenis syndrome, Stickler syndrome, Tay-Sachs disease, Thrombocytopenia Absent Radius (TAR) syndrome, Treacher Collins syndrome, trisomy, tuberous sclerosis, Turner's syndrome, urea cycle disorder, von Hippel-Landau disease, Waardenburg syndrome, Williams syndrome, Wilson's disease, Wiskott-Aldrich syndrome, X-linked lymphoproliferative syndrome (XLP, OMIM No. 308240), acquired immunodeficiencies, lysosomal storage diseases (e.g., Gaucher's disease, GM1, Fabry disease and Tay-Sachs disease), mucopolysaccahidosis (e.g. Hunter's disease, Hurler's disease), hemoglobinopathies (e.g., sickle cell diseases, HbC, α-thalassemia, β-thalassemia) and hemophilias, and Leber's congenital amaurosis (LCA)

The genes that may be integrated or corrected include fibrinogen, prothrombin, tissue factor, Factor V, Factor VII, Factor VIII, Factor IX, Factor X, Factor XI, Factor XII (Hageman factor), Factor XIII (fibrin-stabilizing factor), von Willebrand factor, prekallikrein, high molecular weight kininogen (Fitzgerald factor), fibronectin, antithrombin III, heparin cofactor II, protein C, protein S, protein Z, protein Z-related protease inhibitor, plasminogen, alpha 2-antiplasmin, tissue plasminogen activator, urokinase, plasminogen activator inhibitor-1, plasminogen activator inhibitor-2, glucocerebrosidase (GBA), α-galactosidase A (GLA), iduronate sulfatase (IDS), iduronidase (IDUA), acid sphingomyelinase (SMPD1), MMAA, MMAB, MMACHC, MMADHC (C2orf25), MTRR, LMBRD1, MTR, propionyl-CoA carboxylase (PCC) (PCCA and/or PCCB subunits), a glucose-6-phosphate transporter (G6PT) protein or glucose-6-phosphatase (G6Pase), an LDL receptor (LDLR), ApoB, LDLRAP-1, a PCSK9, a mitochondrial protein such as NAGS (N-acetylglutamate synthetase), CPS1 (carbamoyl phosphate synthetase I), and OTC (ornithine transcarbamylase), ASS (argininosuccinic acid synthetase), ASL (argininosuccinase acid lyase) and/or ARG1 (arginase), and/or a solute carrier family 25 (SLC25A13, an aspartate/glutamate carrier) protein, a UGT1A1 or UDP glucuronsyltransferase polypeptide A1, a fumarylacetoacetate hydrolyase (FAH), an alanine-glyoxylate aminotransferase (AGXT) protein, a glyoxylate reductase/hydroxypyruvate reductase (GRHPR) protein, a transthyretin gene (TTR) protein, an ATP7B protein, a phenylalanine hydroxylase (PAH) protein, a lipoprotein lyase (LPL) protein, an engineered nuclease, an engineered transcription factor and/or a therapeutic single chain antibody and RPE65.

To determine the efficacy of the GEC, both the location of gene editing reagents and frequency of genome edits in target cells can be determined. Location of gene editing reagents, whether in protein, nucleic acid, or viral format, can be determined using any suitable molecular biology methods, including Southern blotting, Western blotting, Northern blotting or polymerase chain reaction. Detecting the frequency of genome edits can be determined using any suitable molecular or cell biology method, including polymerase chain reaction, fluorescent markers, or Southern blotting.

To determine the efficacy of the S-GEC, the concentration of gene editing reagents in systemic fluid or organs can be determined. Suitable detection methods include real-time polymerase chain reaction, digital polymerase chain reaction, branched chain amplification, Western blotting, Southern blotting, Northern blotting, or enzyme-linked immunosorbent assay.

In certain embodiments, the AAV vectors as described herein can be derived from any AAV. In certain embodiments, the AAV vector is derived from the defective and nonpathogenic parvovirus adeno-associated type 2 virus. All such vectors are derived from a plasmid that retains only the AAV 145 bp inverted terminal repeats flanking the transgene expression cassette. Efficient gene transfer and stable transgene delivery due to integration into the genomes of the transduced cell are key features for this vector system. (Wagner et al., Lancet 351:9117 1702-3, 1998; Kearns et al., Gene Ther. 9:748-55, 1996). Other AAV serotypes, including AAV1, AAV2, AAV3, AAV4, AAV5, AAV6, AAV7, AAV8, AAV9 and AAVrh.10 and any novel AAV serotype can also be used in accordance with the present invention. In some embodiments, chimeric AAV is used where the viral origins of the long terminal repeat (LTR) sequences of the viral nucleic acid are heterologous to the viral origin of the capsid sequences. Non-limiting examples include chimeric virus with LTRs derived from AAV2 and capsids derived from AAV5, AAV6, AAV8 or AAV9 (i.e. AAV2/5, AAV2/6, AAV2/8 and AAV2/9, respectively).

Retroviral vectors include those based upon murine leukemia virus (MuLV), gibbon ape leukemia virus (GaLV), Simian Immunodeficiency virus (SIV), human immunodeficiency virus (HIV), and combinations thereof (see, e.g., Buchscher et al., J. Virol. 66:2731-2739, 1992; Johann et al., J. Virol. 66:1635-1640, 1992; Sommerfelt et al., Virol. 176:58-59,1990; Wilson et al., J. Virol. 63:2374-2378, 1989; Miller et al., J. Virol. 65:2220-2224, 1991).

The constructs described herein may also be incorporated into an adenoviral vector system. Adenoviral based vectors are capable of very high transduction efficiency in many cell types and do not require cell division. With such vectors, high titer and high levels of expression can been obtained.

Replication-deficient recombinant adenoviral vectors (Ad) can also be used with the polynucleotides described herein. Most adenovirus vectors are engineered such that a transgene replaces the Ad E1a, E1b, and/or E3 genes; subsequently the replication defective vector is propagated in human 293 cells that supply deleted gene function in trans. Ad vectors can transduce multiple types of tissues in vivo, including nondividing, differentiated cells such as those found in liver, kidney and muscle. Conventional Ad vectors have a large carrying capacity. An example of the use of an Ad vector in a clinical trial involved polynucleotide therapy for antitumor immunization with intramuscular injection (Sterman et al., Hum. Gene Ther. 7:1083-9, 1998). Additional examples of the use of adenovirus vectors for gene transfer in clinical trials include Rosenecker et al., Infection 24:1 5-10, 1996; Sterman et al., Hum. Gene Ther. 9:7 1083-1089, 1998; Welsh et al., Hum. Gene Ther. 2:205-1, 1995; Alvarez et al., Hum. Gene Ther. 5:597-613, 1997; Topf et al., Gene Ther. 5:507-513, 1998; Sterman et al., Hum. Gene Ther. 7:1083-1089, 1998.

The invention will be further described in the following examples, which do not limit the scope of the invention described in the claims.

### EXAMPLES

### Example 1—Design of Catheters for the Delivery and Capture of Gene Editing Reagents

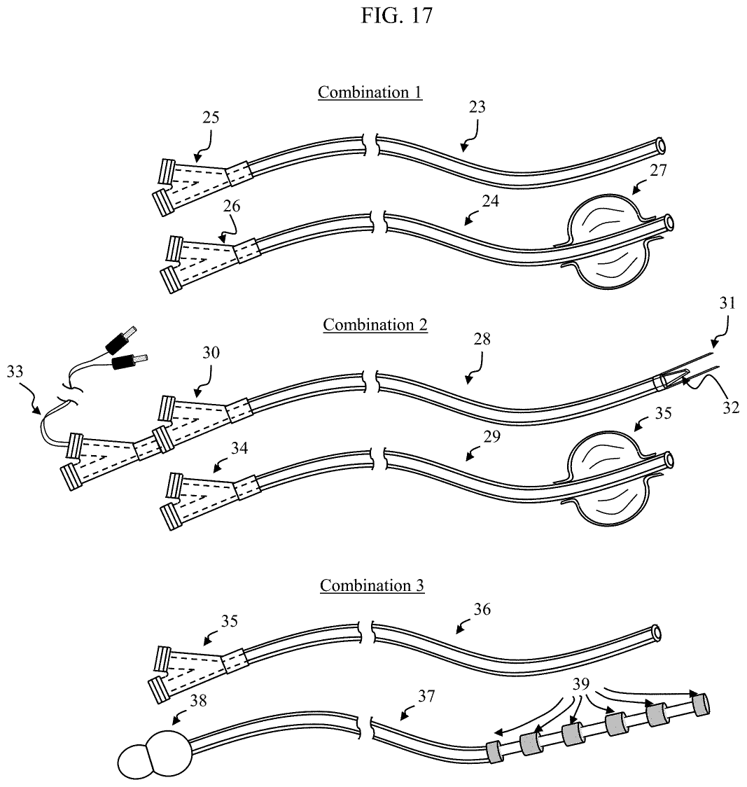

Three sets of catheters were designed to deliver and capture gene editing reagents (FIG. 17). The first set, designated combination 1, comprised a delivery catheter (23) and collection catheter (24; FIG. 17). The delivery catheter comprised an inner material of 63D Pebax SA01 MED, a polycarbonate hub (25), Loctite AA3311 bonding adhesive, an overall length of 19.9 inches, an outer diameter of 0.092 inches, an inner diameter of circular channel of 0.030 inches, and an inner diameter of semi-circular channel at the widest point of 0.025 inches. The collection catheter comprised an inner material of 63D Pebax SA01 MED, a polycarbonate hub (26), Loctite AA3311 bonding adhesive, a urethane balloon (27), an overall length of 19.3 inches, a length of balloon from distal tip to second bond of 2 inches, an outer diameter of the distal balloon bond of 0.1365 inches, an outer diameter of proximal balloon bond of 0.1340 inches, an outer diameter shaft of 0.1195 inches, an inner diameter of circular channel of 0.035 inches, and an inner diameter of semi-circular channel at widest point of 0.025 inches.

The second set of catheters, designated combination 2, comprised a delivery catheter (28) with electrodes and a collection catheter (29; FIG. 17). The delivery catheter with electrodes comprised an inner material of 63D Pebax SA01 MED, an outer material of 63D Pebax and 70D Pebax, a hub of polycarbonate (30) and a silicon tuohy seal, a solid copper motor wire, two stainless steel subdermal needle electrodes (31), a polyimide delivery tip positioned between the two needle electrodes (32), 22-gauge coated wire (33), an overall length of 16.5 inches, a length of needle electrodes from distal end of 0.6035 inches, a length of the polyamide delivery tip from distal end of 0.246 inches, a distance between the two needle electrodes of 0.0745 inches, an outer diameter of catheter shaft of 0.1095 inches, a length of sheath of 13.8 inches, an outer diameter of the sheath of 0.1830 inches, an inner diameter of the sheath of 0.170 inches, a length of opening in sheath of 5.57 inches, an inner diameter of circular channel of 0.030 inches, an inner diameter of semi-circular channel at widest point of 0.025 inches. The collection catheter comprised an inner material of 63D Pebax SA01 MED, a polycarbonate hub (34), Loctite AA3311 bonding adhesive, a urethane balloon (35), an overall length of 19.3 inches, a length of balloon from distal tip to second bond of 2 inches, an outer diameter of the distal balloon bond of 0.1365 inches, an outer diameter of proximal balloon bond of 0.1340 inches, an outer diameter shaft of 0.1195 inches, an inner diameter of circular channel of 0.035 inches, and an inner diameter of semi-circular channel at widest point of 0.025 inches.

The third set of catheters, designated combination 3, comprised a delivery catheter (36) and a magnetic capturing catheter (37; FIG. 17). The delivery catheter comprised an inner material of 63D Pebax SA01 MED, a polycarbonate hub (35), Loctite AA3311 bonding adhesive, an overall length of 19.9 inches, an outer diameter of 0.092 inches, an inner diameter of circular channel of 0.030 inches, and an inner diameter of semi-circular channel at the widest point of 0.025 inches. The magnetic capturing catheter comprised an inner material of 63D Pebax SA01 MED, an outer material of 63D Pebax SA01 MED, a hub of polycarbonate (38) and a silicone Tuohy seal, Loctite AA3311 and 4011 bonding adhesive, neodymium disc magnets (39), an overall length of 19 inches, an overall length of magnetic section of 2.5745 inches, an outer diameter of magnets of 0.25 inches, a length of the first and last magnets of 0.127 inches, a length of middle magnets of 0.2475 inches, an outer diameter of blue Pebax sections of 0.132 inches, a length of blue Pebax sections of 0.263 inches, an outer diameter of catheter shaft of 0.1195 inches, an inner diameter of the circular channel of 0.040 inches, and an inner diameter of semi-circular channel at widest point of 0.030 inches.

### Example 2—Delivery of Gene Editing Reagents within a Closed-Loop Perfusion Circulation System

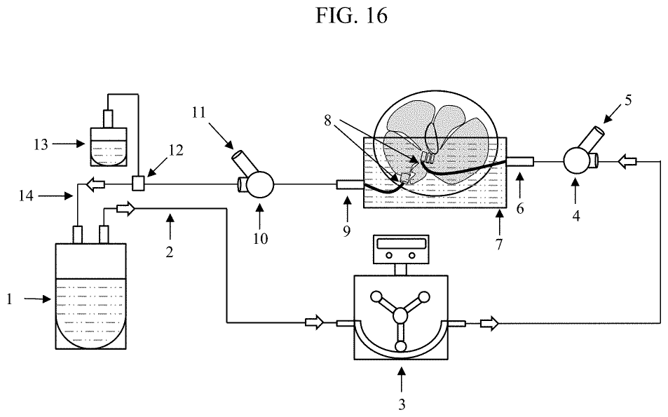

To create an ex vivo system capable of maintaining an organ in an in vivo-like state, a closed-loop perfusion system was designed (FIG. 16). The system comprised a peristaltic pump (Masterflex; L/S Easy Load II, Thin Wall), tubing (platinum-cured silicone tubing; size 18; 0.38 to 2300 mL/min; 8 mm I.D.), valves for depositing gene editing reagents or inserting catheters, a reservoir for holding an organ, and a reservoir comprising perfusion solution. The perfusion solution pumped through the system was histidine-thymine-ketogluterate (HTK) solution. The HTK solution was composed of sodium chloride (15 mmol/1), potassium chloride (9.0 mmol/1), magnesium chloride hexahydrate (4.0 mmol/1), histidine hydrochloride monohydrate (18 mmol/1), histidine (180 mmol/1), tryptophan (2.0 mmol/1), mannitol (30 mmol/1), calcium chloride dihydrate (0.015 mmol/1), and potassium hydrogen 2-ketogluterate (1.0 mmol/1), pH 7.2.

To test delivery and capture of gene editing reagents with the catheters, the perfusion system was used without an organ. To mimic certain features of organs, the reservoir for holding the organ was adapted to be an air-tight fluid chamber.

Plasmids were constructed encoding Cas9 nucleases targeting the KIT gene in Sus scrofa. Two plasmids were generated, referred to herein as pBA1170 and pBA1171. Both plasmids comprised a CMV enhancer, chicken beta-actin promoter driving expression a Cas9-GFP coding sequence and a U6 promoter driving expression of one of the two gRNAs. The sequence of the Cas9 cassette is shown in SEQ ID NO:3.

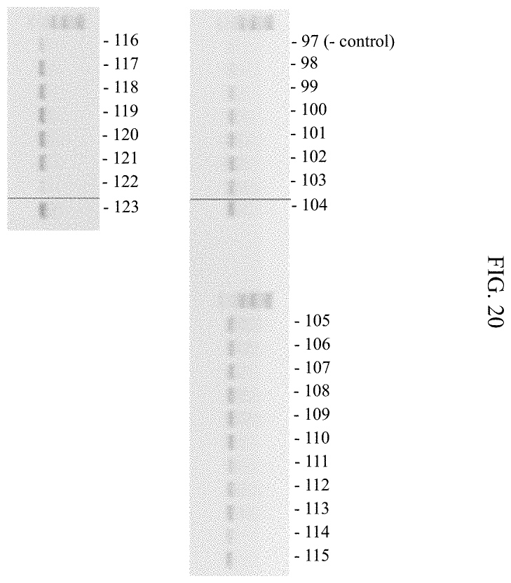

Delivery and capture of gene editing reagents using catheter combination 1 was confirmed using the closed-loop perfusion circulation system. Here, the delivery catheter was inserted into the y-valve on the arterial line (5) and the capturing catheter was inserted into the y-valve in the venous line (11). While HTK solution was being perfused through the system, the delivery catheter delivered 5 mL of gene editing reagents (50 ug of pBA1170, 50 ug of pBA1171, in 5 mL 0.9% saline). Concurrent with delivery, the balloon on the collection catheter was inflated. Fluid exiting the chamber was collected through the channel within the collection catheter. Approximately 300 mL volume was collected. Successful capture of gene editing reagents was confirmed by PCR of the captured fluid (FIG. 20; lanes 115-121).

Delivery and capture of gene editing reagents using catheter combination 3 was confirmed using the closed-loop perfusion circulation system. Here, the delivery catheter was inserted into the y-valve on the arterial line (5) and the capturing catheter was inserted into the y-valve in the venous line (11). While HTK solution was being perfused through the system, the delivery catheter delivered 5 mL of gene editing reagents bound to magnetic nanoparticles (50 ug of pBA1170, 50 ug of pBA1171, 100 ul of magnetic iron oxide core coated with polyethyleneimine (polyMag), 5 mL 0.9% saline). Fluid exiting the chamber passed over the magnet on the end of the capturing catheter. Capture of gene editing reagents with the magnet was confirmed by PCR (FIG. 20 lane 122). Here, the magnet was removed from the catheter and placed in a 1.7 mL centrifuge tube comprising 400 ul of sterile water. The tube was placed in a dry bath, heated to 90° C., and vortexed. 2 ul of solution was used in the PCR.

### Example 3—Delivery of Gene Editing Reagents to Hepatocytes in Swine Livers

Gene editing reagents were designed to target the KIT gene in Sus scrofa. Two Cas9 nucleases were designed to target the genome sequences ACCCTGAGGAGGTAGTTCAA (SEQ ID NO:1) and AGTGGAGGTGATTCTCATGG (SEQ ID NO:2). The target sites were approximately 10.2 kb apart. Successful delivery of both gRNAs to a single cell was anticipated to result in deletion of the sequence between the gRNA target sites, allowing for detecting of gene editing by PCR using primers spanning the intervening sequence (i.e., presence of a band suggests deletion of the intervening sequence). Two gene editing plasmids were generated, pBA1170 and pBA1171, which were the same as used in the experiments described in Example 2.

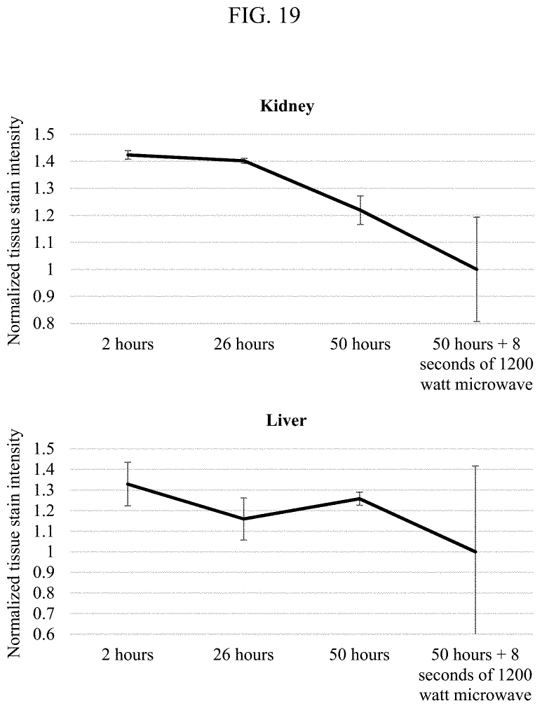

Organs from adult Yorkshire pigs (approximately 220 pounds) were chosen for the experiments. First, cell viability from liver and kidney tissue was determined over the course of 50 hours by trypan blue staining. Immediately following removal, swine livers and kidneys, including surrounding vasculature tissue, were perfused with cold HTK solution (approximately 4 degrees Celsius). Livers and kidneys were placed in a bag containing cold HTK solution, and then the bag was placed in an ice bath. After approximately 2 hours, the organs were removed from the ice bath and stored at room temperature. Sections (approximately 1 cm×1 cm) of the organs were removed and placed in HTK solution (room temperature). Subsets of the sections were taken for trypan blue staining at 2 hours, 26 hours, and 50 hours post-harvest. As a control for cell death, a subset of tissue was exposed to 8 seconds of 1200 watt microwaves. As shown in FIG. 19, the level of blue staining generally increased over the course of 50 hours (i.e., suggesting cell viability decreased over 50 hours). The data suggested the renal cells had faster cell death than the hepatocytes. Overall, the data suggested that i) the viability of kidney and liver cells within HTK solution at room temperature decreases over 50 hours ii) there may be kidney and liver cells still viable after 50 hours, and iii) the optimal time period to deliver gene editing reagents is shortly after harvesting.

To deliver gene editing reagents, Sus scrofa organs were isolated and placed in the perfusion system. Immediately following removal, swine livers and kidneys, including surrounding vasculature tissue, were perfused with cold HTK solution (approximately 4 degrees Celsius). Livers and kidneys were placed in a bag containing cold HTK solution, and then placed in an ice bath. The organs were then connected to the perfusion circuit shown in FIG. 16. For the liver, perfusion of HTK solution proceeded through the portal vein and exited at the hepatic vein/inferior vena cava. To localize the delivery of gene editing reagents, the right lateral lobe (RLL) was isolated by occluding flow to the right medial lobe, left medial lobe, and left lateral lobe by clamping off the portal vein following the branch to the RLL.

Two formats of gene editing reagents were generated: naked plasmid DNA, and plasmid DNA bound to magnetic nanoparticles. The magnetic nanoparticles comprised a magnetic iron oxide core coated with polyethyleneimine (polyMag). Naked plasmid DNA was prepared by combining 1 mg of a 1:1 mixture of pBA1170 and pBA1171 with 6 mL of 0.9% saline. The magnetic nanoparticle/DNA mixture was prepared by combining 1.5 mg of a 1:1 mixture of pBA1170 and pBA1171 with 1.5 mL of polyMag and 22 mL of 0.9% saline.

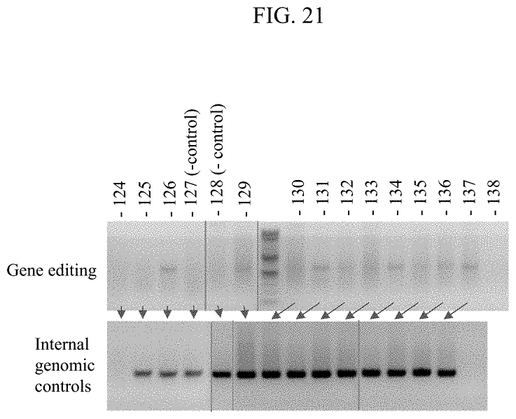

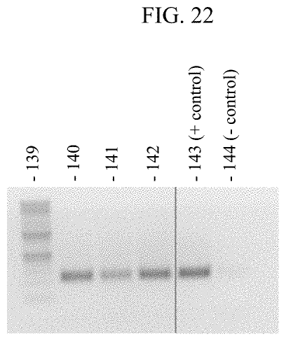

Catheter combination 1 was used for the first experiment in combination with the magnetic nanoparticle-bound gene editing reagents and an external neodymium (N52) magnet. The distal end of the delivery catheter was inserted through the Y-connector in the arterial line (5). The catheter was advanced to the portal vein in proximity to the liver. The distal end of the collection catheter was inserted through the Y-connector in the venous line (11) and placed within the inferior vena cava/hepatic vein lumen. HTK solution was perfused through the liver at approximately 150 mL per minute. A neodymium block magnet (N52; 2 inch by 2 inch by 0.5 inch square block) was positioned beneath the RLL. Gene editing reagents (12.5 mL; 500 ug of both pBA1170 and pBA1171 bound to magnetic nanoparticles), were delivered through the delivery catheter. The balloon on the collection catheter was inflated and fluid exiting the liver was collected (approximately 500 mL). The magnet was kept in contact with the RLL for 30 minutes. Following delivery, the liver was placed in HTK solution and maintained at room temperature for 24 hours. Sections of tissue from the RLL were then removed and assessed for successful delivery of gene editing reagents. DNA was isolated from the liver tissue using NucleoSpin purification. PCR was used with primers to detect gene editing reagents within the tissue of the RLL (FIG. 20; lanes 98-101). Additionally, PCR was used with primers designed to detect the presence of the 10 kb deletion within the KIT gene. As shown in FIG. 21, a band was present in tissue delivered the gene editing reagents and exposed to the magnet (126), whereas no band was present in tissue delivered the gene editing reagents but not exposed to the magnet (127). Fluid that exited the liver and was captured by the collection catheter was analyzed for the presence of gene editing reagents. In one sample, plasmid DNA from 400 ul of fluid was purified using NucleoSpin columns. In a second sample, plasmid DNA from 4 mL of fluid was purified using NucleoSpin columns. PCR was performed on the purified products using primers designed to detect the gene editing plasmid DNA. As shown in FIG. 22, gene editing reagents were detected in the fluid captured by the collection catheter (lanes 140-142).

Catheter combination 3 was used for the next experiment in combination with magnetic nanoparticle bound gene editing reagents. The distal end of the delivery catheter was inserted through the Y-connector in the arterial line (5) in proximity to the portal vein. The distal end of the magnetic collection catheter was inserted through the Y-connector in the venous line (11) and placed in the inferior vena cava lumen in proximity to the liver. HTK solution was perfused through the liver at approximately 150 mL per minute. Gene editing reagents, (12.5 mL; 500 ug of both pBA1170 and pBA1171 bound to magnetic nanoparticles), were dispensed through the delivery catheter. Following delivery, the liver was placed in HTK solution and maintained at room temperature for 24 hours. Further, the magnets on the collection catheter were collected and stored at −20° C. The surface of the neodymium magnets were analyzed for successful capture of gene editing reagents. Here, the distal magnet was removed from the catheter and placed in a 1.7 mL centrifuge tube comprising 400 ul of sterile water. The tube was placed in a dry bath, heated to 90° C., and vortexed. 2 ul of solution was used in the PCR. The results suggest that gene editing reagents were captured by the collection catheter (FIG. 20; lane 114).

Catheter combination 2 was used for the next experiments in combination with gene editing reagents in the form of naked, supercoiled DNA. Here, the distal end of the catheter with needle electrodes was navigated down the portal vein and traversed approximately three fourths of the RLL. Once positioned, 2 mL of the gene editing solution was delivered. Immediately following delivery, electric pulses were delivered through the needle electrodes. The needle electrodes were connected to a BTX T820 electro square porator which produced square waves of 200 V/cm pulse, 20 ms duration, and 10 pulses. Following electroporation, the liver was placed in HTK solution and maintained at room temperature for 24 hours. Sections of tissue from the RLL near the location of the needle electrodes are removed an assessed for successful delivery of gene editing reagents.

To assess the utility of external delivery of gene editing reagents to organs—kidneys were delivered gene editing reagents and exposed to external electrical pulses. Kidneys were directly injected with approximately 40 ug of pBA1170 and pBA1171 and subjected to electrical pluses using needle electrodes. The needle electrodes were connected to a BTX T820 electro square porator which produced square waves of 200 V/cm pulse, 20 ms duration, and 10 pulses. Following electroporation, kidneys were stored in HTK solution and maintained at room temperature for 24 hours. Sections of renal tissue encompassing the electroporated tissue were analyzed for the presence of gene editing reagents and for gene editing. As a control, tissue neighboring an electroporated site was taken. As shown in FIG. 20 (lanes 105-113), gene editing reagents were verified to be present within the renal tissue, including the control (lane 104). The gene editing reagents within the control suggests that DNA may have migrated from the injection sites into the neighboring tissue. For detecting gene editing, PCR was used to detect the presence of a 10 kb deletion. In tissue delivered gene editing reagents and electroporated, bands were observed (FIG. 21; lanes 129-137). In control tissue neighboring electroporation sites, no band was present (lane 128).

### Example 4—Delivery of Gene Editing Reagents to Hepatocytes in Swine

Gene editing reagents for the modification of hepatocytes are designed to be carried by adeno-associated viral (AAV) particles. Gene editing reagents are designed to knock-in an NLS-tagged GFP marker downstream of the endogenous apolipoprotein A2 (APOA2) gene. Two AAV vectors are designed to harbor the gene editing reagents. The first AAV vector encodes SpCas9 (referred to as AAV-SpCas9), while the second AAV vector encodes the associated gRNA driven by a U6 promoter and donor molecule for targeted integration of GFP (referred to as AAV-SpGuide+Donor). High titer AAV1/2 particles are produced using AAV1 and AAV2 serotype plasmids at equal ratios. HEK293FT cells are transfected with the plasmid of interest, pAAV1 plasmid, pAAV2 plasmid, helper plasmid pDF6, and PEI Max in Dulbecco's modified Eagle medium. At 72 hr post transfection, the cell culture media is discarded. Then the cells are rinsed and pelleted via low-speed centrifugation. Afterward, the viruses are applied to HiTrap heparin columns and washed with a series of salt solutions with increasing molarities. During the final stages, the eluates from the heparin columns are concentrated using Amicon ultra-15 centrifugal filter units. Titering of viral particles is executed by quantitative PCR using custom Cre-targeted Taqman probes.

Two locations are chosen for gene editing reagent deposition: the portal vein and hepatic artery (FIG. 8).

Positioning of a catheter within the portal vein in swine is achieved through percutaneous transsplenic portal vein catheterization. Using ultrasonic guidance, a 20-cm-long 1.3-mm-diameter needle is used to puncture subcostally a splenic vein near the plenichilum. By the Seldinger technique, a curved hydrophilic 0.9-mm guide wire and a 1.35-mm catheter are advanced into the splenic vein. The catheter is then advanced to the portal vein, where the solution containing the gene editing reagents is dispensed.

Positioning of a catheter within the hepatic artery is achieved by entering through the left axillary artery. A branch of the axillary artery, specifically the thoracoacromial artery, is surgically exposed under the left clavicle, and a 5-French, 30-cm-long introducer sheath is inserted through this branch and into the descending aorta. A catheter is then inserted through this access route through the celiac trunk and into the hepatic artery where the gene editing reagents are dispensed.

One day post delivery, the liver is removed and assessed for targeted knock-in of GFP. Tissue from the liver is embedded in standard paraffin and sectioned using a microtome. Tissue is analyzed for presence of GFP. Observation of cells containing GFP shows successful gene editing of liver cells. Additional tissue is used for PCR and sequencing. DNA is extracted using conventional methods. The DNA is then used as a template in PCR reactions using primers specific for the APOA2-GFP knocking event. The presence of PCR bands of the expected size and sequence indicates successful knock-in of the GFP gene.

Together, these results show the use of catheters to site-specifically deposit gene editing reagents in the liver.

### Example 5—Capture of Gene Editing Reagents Exiting the Liver

To reduce the systemic spread of gene editing reagents exiting the liver, a second device is positioned downstream of the liver. Two methods are used to capture gene editing reagents: (i) a collection device is placed within the left, right and middle hepatic veins to divert the blood exiting the liver to a collection bin or (ii) a device with a magnet is positioned in the inferior vena cava following the connection of the hepatic arteries. Here, a diametrically magnetized ring is positioned in the inferior vena cava.

Gene editing reagents for the modification of the genome within liver cells are generated in the form of AAV1/2 particles vectors encoding Cas9 and donor molecules (AAV-Cas9 and AAV-SpGuide+Donor). The AAV particles are then combined with biodegradable cationic magnetic nanoparticles. In addition to AAV particles, gene editing reagents are generated in the form of naked and supercoiled DNA (SpCas9, donor molecule and gRNA). The final plasmids are then combined with biodegradable cationic magnetic nanoparticles.

Before the GEC is positioned within the hepatic vein or portal vein, the S-GEC comprising a collection apparatus or magnet is positioned is positioned in the left, right and middle hepatic veins or the inferior vena cava. The GEC is then positioned and gene editing reagents are dispensed. Reduced systemic spread of gene editing reagents is determined using high pressure liquid chromatography—size exclusion chromatography, real time PCR and enzyme-linked immunosorbent assay of blood that has passed through the heart. A reduced amount of Cas9 nucleic acids or AAV particles in systemic blood, compared to a control where an S-GEC is not used, indicates functionality of the gene editing reagent collection system.

### Example 6—Delivery of Cas9 RNP and Donor Molecules to Liver Cells

Gene editing reagents for the modification of the genome within liver cells are generated in the form of purified protein (SpCas9), RNA (gRNA) and naked DNA (donor molecule). Cas9 protein is generated by overexpression and purification from bacteria. To this end, Cas9 protein is expressed with a N-terminal hexahistidine tag and maltose binding protein in E. coli Rosetta 2 cells. The His tag and maltose binding protein are cleaved by TEV protease, and Cas9 is purified. Cas9 protein is stored in 20 mM 2-[4-(2-hydroxyethyl)piperazin-1-yl]ethanesulfonic acid (HEPES) at pH 7.5, 150 mM KCl, 10% glycerol, 1 mM tris(2-chloroethyl) phosphate (TCEP) at −80° C. The corresponding gRNA targeting APOA2 is generated by T7 in vitro transcription.

Cas9 RNP is prepared shortly before delivery by incubating Cas9 protein with sgRNA at 1:1.2 molar ratio in 20 mM HEPES (pH 7.5), 150 mM KCl, 1 mM MgCl₂, 10% glycerol and 1 mM TCEP at 37° C. The donor molecule is then added to the RNP mixture.

In addition to having a reagent dispensing capability, the GEC is designed to harbor an electrode with the ability to generate electrical pulses. Two locations are chosen for gene editing reagent deposition: the portal vein and hepatic artery. Positioning of the catheter within the portal vein and hepatic artery is achieved using the methods described in Example 1. Once positioned, gene editing reagents are dispensed followed by delivery of electric pulses. Electrical pulses are delivered with a variable waveform modulator with a wide dynamic range and bandwidth.

One day post delivery, the liver is removed and assessed for targeted knock-in of GFP. Tissue from the liver is embedded in standard paraffin and sectioned using a microtome. Tissue is analyzed for presence of GFP. Observation of cells containing GFP suggests successful gene editing of liver cells. A higher frequency of GFP in samples administered electrical pulses indicates that the GEC with an electrode can facilitate cellular uptake of gene editing reagents. Additional tissue is used for PCR and sequencing. DNA is extracted using conventional methods. The DNA is then used as a template in PCR reactions using primers specific for the APOA2-GFP knocking event. The presence of PCR bands of the expected size and sequence indicate successful knock-in of the GFP gene.

### Example 7—Delivery of Gene Editing Reagents to Pancreas Cells in Swine

Gene editing reagents for the modification of pancreas cells are designed to knock-in a GFP marker downstream of the chymotrypsin like elastase family member 3A (CELA3A) gene. Gene editing reagents are generated in the form of purified protein (SpCas9), RNA (gRNA) and naked DNA (donor molecule). Cas9 protein is generated by overexpression and purification from bacteria. To this end, Cas9 protein is expressed with an N-terminal hexahistidine tag and maltose binding protein in E. coli Rosetta 2 cells. A GST tag is placed on the C-terminus to facilitate capture by an S-GEC. The His tag and maltose binding protein are cleaved by TEV protease, and the GST-tagged Cas9 is purified. Cas9 protein is stored in 20 mM 2-[4-(2-hydroxyethyl)piperazin-1-yl]ethanesulfonic acid (HEPES) at pH 7.5, 150 mM KCl, 10% glycerol, 1 mM tris(2-chloroethyl) phosphate (TCEP) at −80° C. The corresponding gRNA targeting the 3′ UTR of the CCM2 like scaffolding protein (CCM2L) gene is generated by T7 in vitro transcription.

Cas9 RNP is prepared shortly before delivery by incubating Cas9 protein with sgRNA at 1:1.2 molar ratio in 20 mM HEPES (pH 7.5), 150 mM KCl, 1 mM MgCl₂, 10% glycerol and 1 mM TCEP at 37° C. The donor molecule is then added to the RNP mixture.

Before placement of the GEC, an S-GEC is positioning within the portal vein. Placement is achieved through percutaneous transsplenic portal vein catheterization. Using ultrasonic guidance, a 20-cm-long 1.3-mm-diameter needle is used to puncture subcostally a splenic vein near the plenichilum. By the Seldinger technique, a curved hydrophilic 0.9-mm guide wire and a 1.35-mm catheter are advanced into the splenic vein. The S-GEC comprises glutathione which is designed to capture the purified Cas9 protein exiting the liver.

Two locations are chosen for the GEC: the dorsal pancreatic artery (FIG. 9) and the greater pancreatic artery. Once positioned, gene editing reagents are dispensed followed by delivery of electric pulses. Electrical pulses are delivered with a variable waveform modulator with a wide dynamic range and bandwidth.

One day post delivery, the pancreas is removed and assessed for targeted knock-in of GFP. Tissue from the liver is embedded in standard paraffin and sectioned using a microtome. Tissue is analyzed for presence of GFP. Observation of cells containing GFP shows successful gene editing of liver cells. Additional tissue is used for PCR and sequencing. DNA is extracted using conventional methods. The DNA is then used as a template in PCR reactions using primers specific for the CCM2L-GFP knocking event. The presence of PCR bands of the expected size and sequence indicates successful knock-in of the GFP gene.

Together, these results show the use of catheters to site-specifically deposit gene editing reagents in the pancreas.

Reduced systemic spread of gene editing reagents is determined using high pressure liquid chromatography—size exclusion chromatography, real time PCR and enzyme-linked immunosorbent assay of blood that has passed through the heart. A reduced amount of Cas9 protein in systemic blood, compared to a control where an S-GEC is not used, indicates functionality of the gene editing reagent collection system.

### Example 8—Delivery of Gene Editing Reagents to the Gastrointestinal Tract in Swine

Gene editing reagents for the modification of cells within the gastrointestinal tract are designed to knock-in a GFP marker downstream of the carcinoembryonic antigen related cell adhesion molecule 5 (CEACAM5) gene. Gene editing reagents are generated in the form of supercoiled DNA. Two plasmids are synthesized: the first encodes Cas9, and the second harbors the GFP donor molecule and also encodes a gRNA targeting the 3′ UTR of the CEACAM5 gene. To promote uptake by cells within the gastrointestinal tract, plasmids are conjugated to PEGylated lipoplexes.

Before placement of the GEC, an S-GEC is positioning within the portal vein. Placement is achieved through percutaneous transsplenic portal vein catheterization. Using ultrasonic guidance, a 20-cm-long 1.3-mm-diameter needle is used to puncture subcostally a splenic vein near the plenichilum. By the Seldinger technique, a curved hydrophilic 0.9-mm guide wire and a 1.35-mm catheter are advanced into the splenic vein. The S-GEC comprises a collection tube, which is designed to capture PEGylated lipoplexes leaving the gastrointestinal tract.

Two locations are chosen for the GEC: the superior mesenteric artery following any branches leading to other organs, and the inferior mesenteric artery.

One day post delivery, the colon is removed and assessed for targeted knock-in of GFP. Tissue from the liver is embedded in standard paraffin and sectioned using a microtome. Tissue is analyzed for presence of GFP. Observation of cells containing GFP shows successful gene editing of colon cells. Additional tissue is used for PCR and sequencing. DNA is extracted using conventional methods. The DNA is then used as a template in PCR reactions using primers specific for the CEACAM5-GFP knocking event. The presence of PCR bands of the expected size and sequence indicates successful knock-in of the GFP gene. Together, these results show the use of catheters to site-specifically deposit gene editing reagents in the liver.

Reduced systemic spread of gene editing reagents is determined using high pressure liquid chromatography—size exclusion chromatography, real time PCR and enzyme-linked immunosorbent assay of blood that has passed through the heart. A reduced amount of Cas9 protein in systemic blood, compared to a control where an S-GEC is not used, indicates functionality of the gene editing reagent collection system.

### Example 9—Delivery and Capture of Gene Editing Reagents in the Liver in Humans

Gene editing reagents carried on rAAV2/6 vectors for the modification of hepatocytes are delivered and captured using a delivery catheter and capturing catheter. The delivery catheter is guided to the hepatic artery proper by traversing the Iliac/femoral artery, abdominal aorta, celiac trunk, and common hepatic artery. The capturing catheter is positioned in proximity to the liver by traversing the common iliac vein and inferior vena cava. A balloon is inflated on the capturing catheter to facilitate collection. Gene editing reagents are dispensed through the delivery catheter. Fluid exiting the liver is collected and disposed.

### Example 10—Delivery and Capture of Gene Editing Reagents in the Kidney in Humans

Gene editing reagents carried on magnetic nanoparticles for the modification of kidney cells are delivered and captured using a delivery catheter and capturing catheter. An external magnet is applied to facilitate transfection. The depositing catheter is guided to the renal artery by traversing the Iliac/femoral artery, and abdominal aorta. Alternatively, the depositing catheter is guided to the renal artery by traversing through the subclavian artery, thoracic aorta, and abdominal aorta. The capturing catheter is positioned in proximity to the kidney by traversing the common iliac vein, and inferior vena cava. Gene editing reagents are dispensed through the delivery catheter. Fluid exiting the liver is collected and disposed.

What is claimed is:

1) A method to deliver gene editing reagents to cells in an organ, the method comprising:
a. selecting a composition comprising at least one gene editing reagent,
b. inserting a first medical device into a first body part that is in fluid communication with said organ,
c. inserting a second medical device into a second body part that is in fluid communication with said organ,
d. administering said composition through said first medical device.

2) The method of claim 1, wherein the first body part is a lumen that is in proximity to or within said organ.

3) The method of claim 2, wherein the first body part is an arterial lumen.

4) The method of claim 3, wherein the second body part is a lumen that is in proximity to or within said organ.

5) The method of claim 4, wherein the first body part is a venous lumen.

6) The method of claim 5, wherein said first and second medical devices are catheters.

7) The method of claim 6, wherein the first or second medical device further comprises an accessory selected from the group consisting of a balloon, electrode, magnet, needle, or acoustic device.

8) The method of claim 1, wherein the organ is selected from the group consisting of the liver, pancreas, spleen, gastrointestinal tract, brain, lungs, prostate, eye, kidney and heart.

9) The method of claim 5, wherein the organ is the liver and the first medical device is inserted into the hepatic artery and the second medical device is inserted into a hepatic vein or the inferior vena cava.

10) The method of claim 5, wherein the organ is the kidney and the first medical device is inserted into the renal artery and the second medical device is inserted into the renal vein.

11) The method of claim 8, wherein the organ is from a host selected from the group consisting of a human, mouse, rat, guinea pig, hamster, dog, pig, sheep, chimpanzee, monkey, horse and cow.

12) The method of claim 1, wherein the gene editing reagent is a rare-cutting endonuclease, a transposase, or a donor molecule.

13) The method of claim 12, wherein the at least one gene editing reagent is selected from a CRISPR nuclease, Cas9, Cas12a, transcription activator-like effector nuclease, zinc-finger nuclease, CRISPR-associated transposase, Cas12k, Cas6, transposon, or donor molecule.

14) The method of claim 13, wherein the at least one gene editing reagent is in the form of a protein, a nucleic acid or a virus particle.

15) The method of claim 14, wherein the gene editing reagent is encoded on an AAV vector.

16) The method of claim 13, wherein said composition further comprises magnetic nanoparticles or lipid nanoparticles.

17) The method of claim 16, wherein said composition comprises a CRISPR nuclease or transposase and a magnetic nanoparticle.

18) The method of claim 16, wherein said composition comprises a CRISPR nuclease or transposase and a lipid nanoparticle.

19) The method of claim 13, wherein the CRISPR nuclease in encoded on one or more RNA molecules or is a ribonucleoprotein.

20) The method of claim 1, further comprising administering an electrical pulse, sound energy or a magnetic field to said organ.

21) The method of claim 6, wherein the second medical device captures or inactivates the gene editing reagents exiting the organ.

22) The method of claim 21, wherein the second medical device comprises a channel to remove fluid exiting the organ.

23) The method of claim 22, wherein the second medical device comprises a balloon and channel to remove fluid exiting the organ.

24) The method of claim 23, wherein the gene editing reagents within the collected fluid are removed and the fluid is re-introduced into the host.

25) The method of claim 6, wherein the second medical device comprises a magnet.

26) The method of claim 6, wherein the second medical device delivers a compound that inactivates the gene editing reagent, wherein the compound is selected from a restriction endonuclease, DNase, RNase, RNA oligonucleotide, or anti-CRISPR protein.

27) The method of claim 6, where the inserting of both the first and second medical devices is facilitated with a guidewire.

28) The method of claim 1, wherein the gene editing reagent targets SEQ ID NO:1 or SEQ ID NO:2.

29) A method to deliver gene editing reagents to cells in an organ, the method comprising:
a. selecting a composition comprising at least one gene editing reagent,
b. identifying the organ that has been isolated or removed from a host,
c. connecting said organ to a perfusion system, and
d. administering said gene editing solution to said organ.

30) A kit, comprising:
a. a catheter for delivery of at least one gene editing reagent,
b. a solution comprising at least one gene editing reagent, and
c. optionally, instructions.


SEQUENCE LISTING (3 sequences)

SEQ ID NO: 1 - DNA, 20 bp, Sus scrofa
accctgaggaggtagttcaa

SEQ ID NO: 2 - DNA, 20 bp, Sus scrofa
agtggaggtgattctcatgg

SEQ ID NO: 3 - DNA, 6103 bp, Artificial Sequence
  Cas9 expression cassette
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccgggggggggggggggggggggggggggggggggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaaggaattcggcagtggagagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctgggga